(19) United States
(12) Patent Application Publication
(10) Pub. No.: US 2026/0185043 A1
(43) Pub. Date: Jul. 2, 2026

(54) CONSTRUCTION METHOD AND USE OF GASTRULOID STEM CELL LINE

(71) Applicant: NANJING MEDICAL UNIVERSITY, Nanjing, Jiangsu (CN)

(72) Inventors: Jiahao SHA, Nanjing (CN); Yan YUAN, Nanjing (CN); Mingqian HUANG, Nanjing (CN); Mengqi CHEN, Nanjing (CN); Hao ZHANG, Nanjing (CN); Gege YUAN, Nanjing (CN)

(73) Assignee: NANJING MEDICAL UNIVERSITY, Nanjing, Jiangsu (CN)

(21) Appl. No.: 19/113,368

(22) PCT Filed: Dec. 29, 2022
(86) PCT No.: PCT/CN2022/143644

§ 371 Date: Feb. 27, 2026

(30) Foreign Application Priority Data
Sep. 19, 2022 (CN) 202211135955.9

(51) Int. Cl. C12N 5/0735

(52) U.S. Cl. CPC C12N 5/0606; C12N 2500/02; C12N 2500/32; C12N 2501/11; C12N 2501/115; C12N 2501/125; C12N 2501/155; C12N 2501/16; C12N 2501/235; C12N 2501/415; C12N 2501/70; C12N 2501/727; C12N 2501/73; C12N 2503/02; C12N 2513/00

(57) ABSTRACT

A construction method and use of a gastruloid stem cell line. Gastruloid stem cells that can be passaged stably are established in this research. The cells maintain the pluripotency of stem cells to a certain extent, exhibit gene and protein expression of cells from three germ layers, endoderm, mesoderm, and ectoderm, during gastrulation, and have characteristics consistent with those of cells during gastrulation, so that key characteristics of cells during gastrulation can be well reproduced. A three-dimensional gastruloid model that can mimic gastrulation can be constructed in mice or in vitro by using the stem cell line, and key biological events can be partially reproduced, so that key characteristics of post-implantation embryos during gastrulation can be well reproduced. Through this model, an in vitro platform for screening drugs affecting early embryonic development can be established, providing a reference for clinical medication.

### TECHNICAL FIELD

The present invention belongs to the field of biotechnologies, and relates to a construction method and use of a gastruloid stem cell line, and specifically, to a method for constructing a gastruloid stem cell line, a gastruloid stem cell line obtained by using the method, and construction and use of a model derived from the cell line.

### BACKGROUND

During human embryogenesis, a fertilized ovum forms tissues and organs through cell division, proliferation, and specialization, and finally forms a highly complex body. Embryonic development during the first three weeks after fertilization is an important stage of human embryogenesis, especially from peri-implantation to gastrulation. At this stage, cells undergo lineage specialization and rearrangement, forming an early embryo. Problems at this stage of development can lead to miscarriage or birth defects. Understanding early development mechanisms of humans is important for developmental biology and regenerative medicine. Due to technical and ethical limitations, limited sample quantities, and other reasons, our research on early embryonic development of humans is incomplete. Most of what we know about early embryonic development of humans today is from studies on histological and anatomical structures of Carnegie-staged embryos, and there are still many uncharted areas of this process that need to be explored.

At present, a breakthrough has been achieved in the study of human pre-implantation and peri-implantation embryonic development. Researchers have been able to culture human embryos in vitro up to day 14 of pre-gastrulation embryos, or induce human pluripotent stem cells (hPSCs) into pre-implantation blastoids, which, in combination with single-cell multi-omics sequencing and fluorescence imaging, has opened new avenues for studying human embryonic development and greatly expanded the understanding of characteristics and mechanisms of human embryonic development from implantation to pre-gastrulation.

Some studies have attempted to establish a three-dimensional gastruloid model using hPSCs to mimic the mutually exclusive separation of cells from three germ layers during gastrulation. However, this model lacks key embryonic structures (bilaminar embryonic disc, amniotic cavity, and yolk sac) and no neural cell lineage differentiation has been observed. In this case, an ideal model for gastruloid research has not been established.

Therefore, there is an urgent need for a method for constructing gastruloid stem cells that can be passaged stably to obtain gastruloid stem cells that can maintain the pluripotency of stem cells, exhibit gene and protein expression of cells from three germ layers, endoderm, mesoderm, and ectoderm, during gastrulation, and have characteristics consistent with those of cells during gastrulation, so that key characteristics of cells during gastrulation are well reproduced and an ideal model for human embryo research is provided.

### SUMMARY

To resolve the foregoing technical problems in the related art, gastruloid stem cells that can be passaged stably are established in this research. The gastruloid stem cells maintain the pluripotency of stem cells to a certain extent, exhibit gene and protein expression of cells from three germ layers, endoderm, mesoderm, and ectoderm, during gastrulation, and have characteristics consistent with those of cells during gastrulation, so that key characteristics of cells during gastrulation can be well reproduced.

Through induced differentiation of the gastruloid stem cells, a three-dimensional gastruloid model that can mimic gastrulation can be constructed in vitro or in mice, and key biological events such as lineage separation of endoderm and ectoderm, formation of proamniotic cavity, emergence of primitive streak, and mesodermal lineage specialization during embryonic development in vivo can be partially reproduced. In combination with single-cell multi-omics sequencing and fluorescence imaging, the key biological events are validated at both protein and transcriptome levels, so that key characteristics of post-implantation embryos during gastrulation can be well reproduced. Through this model, an in vitro platform for screening drugs affecting early embryonic development can be established, providing a reference for clinical medication.

Further, through induced differentiation of the gastruloid stem cells, an organoid model for a tissue or organ from ectoderm, mesoderm, and endoderm, such as neuroepithelium, smooth muscle, or intestines, can be formed.

A first objective of the present invention is to provide a method for constructing the gastruloid stem cell line.

The construction method includes the following steps:
(1) induction from stem cells to mesoderm:
(1-1) digesting stem cells into single cells, centrifuging the cells, and resuspending the cells, to obtain a cell suspension 1, where the stem cells are human embryonic stem cells or human induced pluripotent stem cells, and the human embryonic stem cells are established human embryonic stem cells derived from an embryo that is within 14 days after fertilization and that is not developed in vivo;
(1-2) centrifuging the cell suspension 1, removing a supernatant, and adding a GK15-1 medium containing a ROCK inhibitor to further resuspend cells, to obtain a cell suspension 2, where
preferably, in step (1-2), the cell suspension 2 and the GK15-1 medium containing the ROCK inhibitor are in the following proportion relationship: 1×10⁶ cells per 1 mL of the GK15-1 medium containing the ROCK inhibitor;
(1-3) inoculating the cell suspension 2 obtained in (1-2) into a well plate pre-coated with matrigel for culture, where
preferably, in step (1-3), the cell suspension 2 is inoculated in a density of 0.6-1×10⁵ cells/cm², and
preferably, in step (1-3), the culture is carried out at 37° C. under carbon dioxide in a volume concentration of 5.0%-5.2%, more preferably, the volume concentration of carbon dioxide is 5.0%; and
(1-4) replacing the spent medium with a GK15-1 medium on day 2 of the culture, and changing the medium daily, until nascent mesoderm-like cells are obtained;
(2) induction from the nascent mesoderm-like cells to primordial germ cell-like cells:
(2-1) digesting the nascent mesoderm-like cells obtained in (1-4) into single cells when the cells grow to 60%-90% confluency, centrifuging the cells, and resuspending the cells, to obtain a cell suspension 3;
(2-2) centrifuging the cell suspension 3, removing a supernatant, and adding a GK15-2 medium containing a ROCK inhibitor to further resuspend cells, to obtain a cell suspension 4, where
preferably, in step (2-2), the cell suspension 3 and the GK15-2 medium containing the ROCK inhibitor are in the following proportion relationship: 1×10⁵ cells per 1 mL of the GK15-2 medium containing the ROCK inhibitor; and
(2-3) inoculating the cell suspension 4 obtained in (2-2) into a low-attachment well plate for sphere-forming culture; replacing the spent medium with a GK15-2 medium on day 2 of the culture, and changing the medium daily, until cell spheres containing primordial germ cell-like cells are obtained, where
preferably, in step (2-3), each well for the sphere-forming culture initially contains 0.5-1×10⁴ cells;
(3) cell purification:
(3-1) digesting the cell spheres obtained in (2-3) into single cells, and resuspending the cells with a GK15-2 medium, to obtain a cell suspension 5; and
(3-2) separating CD326 and CD49f double-positive cells from the cell suspension 5 obtained in (3-1), and adding a GK10 medium containing a ROCK inhibitor to resuspend the cells, to obtain a cell suspension 6, where
preferably, in step (3-2), 1×10⁵ double-positive cells are contained per 1 mL of the GK10 medium containing the ROCK inhibitor; and
(4) cell expansion:
inoculating the cell suspension 6 obtained in (3-2) into a well plate in which mouse embryo fibroblasts treated with mitomycin C are pre-spread as feeder cells; replacing the medium with a fresh GK10 medium after 24 hours of culture, and changing the medium daily, to obtain the gastruloid stem cell line, where
preferably, in step (4), the cell suspension 6 is inoculated in a density of 0.4-2×10⁴ cells/cm², and
preferably, in step (4), the culture is carried out at 37° C. under carbon dioxide in a volume concentration of 5.0%.

Further, the GK15-1 medium containing the ROCK inhibitor in (1-2) includes the following components:
80%-85% of basal medium GMEM in percentage by volume, 10%-15% of serum replacement KOSR in percentage by volume, 1% of penicillin-streptomycin double antibiotic solution in percentage by volume, 1% of 10 mM non-essential amino acids in percentage by volume, 1% of 200 mM GlutaMAX additive in percentage by volume, 1% of 100 mM sodium pyruvate additive in percentage by volume, 0.1 mM β-mercaptoethanol, 25-200 ng/mL recombinant human activin A, 1-10 μM glycogen synthase kinase-3 (GSK-3) inhibitor CHIR 99021, and 5-20 μM ROCK inhibitor.

In a specific embodiment, the GK15-1 medium containing the ROCK inhibitor in (1-2) includes the following components:
81% of basal medium GMEM in percentage by volume, 15% of serum replacement KOSR in percentage by volume, 1% of penicillin-streptomycin double antibiotic solution in percentage by volume, 1% of 10 mM non-essential amino acids in percentage by volume, 1% of 200 mM GlutaMAX additive in percentage by volume, 1% of 100 mM sodium pyruvate additive in percentage by volume, 0.1 mM β-mercaptoethanol, 50 ng/mL recombinant human activin A, 3 μM glycogen synthase kinase-3 (GSK-3) inhibitor CHIR 99021, and 10 μM ROCK inhibitor.

Further, the GK15-1 medium in (1-4) includes the following components:
80%-85% of basal medium GMEM in percentage by volume, 10%-15% of serum replacement KOSR in percentage by volume, 1% of penicillin-streptomycin double antibiotic solution in percentage by volume, 1% of 10 mM non-essential amino acids in percentage by volume, 1% of 200 mM GlutaMAX additive in percentage by volume, 1% of 100 mM sodium pyruvate additive in percentage by volume, 0.1 mM β-mercaptoethanol, 25-200 ng/ml recombinant human activin A, and 1-10 μM glycogen synthase kinase-3 (GSK-3) inhibitor CHIR 99021.

In a specific embodiment, the GK15-1 medium in (1-4) includes the following components:
81% of basal medium GMEM in percentage by volume, 15% of serum replacement KOSR in percentage by volume, 1% of penicillin-streptomycin double antibiotic solution in percentage by volume, 1% of 10 mM non-essential amino acids in percentage by volume, 1% of 200 mM GlutaMAX additive in percentage by volume, 1% of 100 mM sodium pyruvate additive in percentage by volume, 0.1 mM β-mercaptoethanol, 50 ng/mL recombinant human activin A, and 3 μM glycogen synthase kinase-3 (GSK-3) inhibitor CHIR 99021.

Further, the GK15-2 medium containing the ROCK inhibitor in (2-2) includes the following components:
80%-85% of basal medium GMEM in percentage by volume, 10%-15% of serum replacement KOSR in percentage by volume, 1% of penicillin-streptomycin double antibiotic solution in percentage by volume, 1% of 10 mM non-essential amino acids in percentage by volume, 1% of 200 mM GlutaMAX additive in percentage by volume, 1% of 100 mM sodium pyruvate additive in percentage by volume, 0.1 mM β-mercaptoethanol, 100-500 ng/mL recombinant human bone morphogenetic protein 4, 50-200 ng/mL recombinant human stem cell factor, 1000-5000 U/mL recombinant human leukemia inhibitory factor, 50-250 ng/mL recombinant human epidermal growth factor, and 5-20 μM ROCK inhibitor.

In a specific embodiment, the GK15-2 medium containing the ROCK inhibitor in (2-2) includes the following components:
81% of basal medium GMEM in percentage by volume, 15% of serum replacement KOSR in percentage by volume, 1% of penicillin-streptomycin double antibiotic solution in percentage by volume, 1% of 10 mM non-essential amino acids in percentage by volume, 1% of 200 mM GlutaMAX additive in percentage by volume, 1% of 100 mM sodium pyruvate additive in percentage by volume, 0.1 mM β-mercaptoethanol, 200 ng/mL recombinant human bone morphogenetic protein 4, 100 ng/mL recombinant human stem cell factor, 1000 U/mL recombinant human leukemia inhibitory factor, 50 ng/ml recombinant human epidermal growth factor, and 10 μM ROCK inhibitor.

Further, the GK15-2 medium in (2-3) and (3-1) includes the following components:
80%-85% of basal medium GMEM in percentage by volume, 10%-15% of serum replacement KOSR in percentage by volume, 1% of penicillin-streptomycin double antibiotic solution in percentage by volume, 1% of 10 mM non-essential amino acids in percentage by volume, 1% of 200 mM GlutaMAX additive in percentage by volume, 1% of 100 mM sodium pyruvate additive in percentage by volume, 0.1 mM β-mercaptoethanol, 100-500 ng/mL recombinant human bone morphogenetic protein 4, 50-200 ng/mL recombinant human stem cell factor, 1000-5000 U/mL recombinant human leukemia inhibitory factor, and 50-250 ng/mL recombinant human epidermal growth factor.

In a specific embodiment, the GK15-2 medium in (2-3) and (3-1) includes the following components:
81% of basal medium GMEM in percentage by volume, 15% of serum replacement KOSR in percentage by volume, 1% of penicillin-streptomycin double antibiotic solution in percentage by volume, 1% of 10 mM non-essential amino acids in percentage by volume, 1% of 200 mM GlutaMAX additive in percentage by volume, 1% of 100 mM sodium pyruvate additive in percentage by volume, 0.1 mM β-mercaptoethanol, 200 ng/ml recombinant human bone morphogenetic protein 4, 100 ng/mL recombinant human stem cell factor, 1000 U/mL recombinant human leukemia inhibitory factor, and 50 ng/mL recombinant human epidermal growth factor.

Further, the GK10 medium containing the ROCK inhibitor in (3-2) includes the following components:
80%-85% of basal medium GMEM in percentage by volume, 10% of serum replacement KOSR in percentage by volume, 2.5% of fetal bovine serum FBS in percentage by volume, 1% of penicillin-streptomycin double antibiotic solution in percentage by volume, 1% of 10 mM non-essential amino acids in percentage by volume, 1% of 200 mM GlutaMAX additive in percentage by volume, 1% of 100 mM sodium pyruvate additive in percentage by volume, 0.1 mM β-mercaptoethanol, 10 μM forskolin, 10 μM Rolipram, 50-200 ng/mL recombinant human stem cell factor, and 5-20 M ROCK inhibitor.

In a specific embodiment, the GK10 medium containing the ROCK inhibitor in (3-2) includes the following components:
83.5% of basal medium GMEM in percentage by volume, 10% of serum replacement KOSR in percentage by volume, 2.5% of fetal bovine serum FBS in percentage by volume, 1% of penicillin-streptomycin double antibiotic solution in percentage by volume, 1% of 10 mM non-essential amino acids in percentage by volume, 1% of 200 mM GlutaMAX additive in percentage by volume, 1% of 100 mM sodium pyruvate additive in percentage by volume, 0.1 mM β-mercaptoethanol, 10 μM forskolin, 10 μM Rolipram, 100 ng/ml recombinant human stem cell factor, and 10 μM ROCK inhibitor.

Further, the GK10 medium in (4) includes the following components:
80%-85% of basal medium GMEM in percentage by volume, 10% of serum replacement KOSR in percentage by volume, 2.5% of fetal bovine serum FBS in percentage by volume, 1% of penicillin-streptomycin double antibiotic solution in percentage by volume, 1% of 10 mM non-essential amino acids in percentage by volume, 1% of 200 mM GlutaMAX additive in percentage by volume, 1% of 100 mM sodium pyruvate additive in percentage by volume, 0.1 mM β-mercaptoethanol, 10 μM forskolin, 10 μM Rolipram, and 50-200 ng/ml recombinant human stem cell factor.

In a specific embodiment, the GK10 medium in (4) includes the following components: 83.5% of basal medium GMEM in percentage by volume, 10% of serum replacement KOSR in percentage by volume, 2.5% of fetal bovine serum FBS in percentage by volume, 1% of penicillin-streptomycin double antibiotic solution in percentage by volume, 1% of 10 mM non-essential amino acids in percentage by volume, 1% of 200 mM GlutaMAX additive in percentage by volume, 1% of 100 mM sodium pyruvate additive in percentage by volume, 0.1 mM β-mercaptoethanol, 10 μM forskolin, 10 μM Rolipram, and 100 ng/mL recombinant human stem cell factor.

A second objective of the present invention is to provide a gastruloid stem cell line. The gastruloid stem cell line is constructed by using the foregoing method.

Further, the gastruloid stem cell line is deposited in the China Center for Type Culture Collection, with a culture name of human gastruloid stem cell line CCRM-hGOSC-1 and an accession number of CCTCC NO. C2022114 on a deposition date of Apr. 27, 2022. The gastruloid stem cell line is constructed by using human embryonic stem cells.

Further, the gastruloid stem cell line is deposited in the China Center for Type Culture Collection, with a culture name of human gastruloid stem cell line DYR0100-hGOSC-1 and an accession number of CCTCC NO. C2022115 on a deposition date of Apr. 27, 2022. The gastruloid stem cell line is constructed by using human induced pluripotent stem cells.

A third objective of the present invention is to provide use of the foregoing gastruloid stem cell line in construction of a gastruloid model.

A fourth objective of the present invention is to provide a gastruloid model. The gastruloid model is obtained through induced differentiation of the foregoing gastruloid stem cell line.

A fifth objective of the present invention is to provide a method for constructing a gastruloid model. The construction method is induced differentiation of the foregoing gastruloid stem cell line, and the induced differentiation is induced differentiation in vivo or induced differentiation in vitro.

Further, the induced differentiation in vivo includes the following steps:
resuspending the foregoing gastruloid stem cell line with a GK10 medium to obtain a cell suspension, and injecting the cell suspension into a testis of a mouse for culture for 10-20 days, to obtain the gastruloid model, where
preferably, the mouse is an immunodeficient mouse; and
further preferably, the mouse is a BALB/c nude mouse.

Further, the cell suspension is injected into the testis of the mouse through the vas deferens of the mouse.

Further, an injection amount is 2-8×10⁴ cells for one testis.

Further, the induced differentiation in vitro includes the following steps:
(1) digesting gastruloid stem cells into single cells when the cells grow to 60%-90% confluency, and resuspending the cells with a GK10 medium, to obtain a cell suspension 7; separating CD326 and CD49f double-positive cells from the cell suspension 7; carrying out centrifugation, resuspending the cells with an mTR medium in a concentration of 1.5-1.75×10⁴ cells/mL, and inoculating the cells into a low-attachment well plate with 6.0-7.0×10³ cells per well for culture, until the gastruloid stem cells are assembled to form a three-dimensional structure; and
(2) adding an E6BIN medium without removing the mTR medium in (1) for culture, until an amniotic cavity is formed and mesoendodermal lineage specialization is completed, to obtain the gastruloid model.

Preferably, an addition amount of the E6BIN medium is 50-200 μL/well.

Preferably, in step (1) and step (2), the culture is carried out at 37° C. under carbon dioxide in a volume concentration of 5.0%.

Further, the mTR medium in step (1) includes the following components: 99% of mTeSR™ 1 complete medium in percentage by volume, 1% of penicillin-streptomycin double antibiotic solution in percentage by volume, and 5-20 μM ROCK inhibitor.

In a specific embodiment, the mTR medium in step (1) includes the following components: 99% of mTeSR™ 1 complete medium in percentage by volume, 1% of penicillin-streptomycin double antibiotic solution in percentage by volume, and 10 μM ROCK inhibitor.

Further, the E6BIN medium in step (2) includes the following components: 100% of Essential 6 medium in percentage by volume, 20 ng/mL recombinant human fibroblast growth factor 2, 50 ng/mL recombinant human Noggin protein, and 5 μM IWP-2.

A sixth objective of the present invention is to provide an organoid model. The organoid model is obtained through culture of the foregoing gastruloid stem cell line injected into an animal testis, and an organoid is a tissue or organ from ectoderm, mesoderm, and endoderm.

Preferably, the culture is carried out for 30-90 days.

A seventh objective of the present invention is to provide a method for constructing an organoid model. The construction method includes the following steps: resuspending the foregoing gastruloid stem cell line with a GK10 medium to obtain a cell suspension, and injecting the cell suspension into a testis of a mouse for culture for 30-90 days, to obtain the organoid model, where preferably, the mouse is an immunodeficient mouse; and further preferably, the mouse is a BALB/c nude mouse.

Further, the cell suspension is injected into the testis of the mouse through the vas deferens of the mouse.

Further, 2-8×10⁴ cells are injected into one testis.

Further, a neural ectoderm model, a primordial germ cell model, and/or an amniotic epithelial cell model are/is obtained on day 30 to day 40 of the culture; a neuroepithelial cell model is obtained on day 40 to day 50 of the culture; and an intestinal organoid model, a muscular organoid model, a cartilaginous organoid model, a neural organoid model, and/or a skin organoid model are/is obtained on day 70 to day 90 of the culture.

An eighth objective of this patent is to provide use of the foregoing gastruloid stem cell line, the foregoing gastruloid model, the foregoing organoid model, or a tissue or organ derived from the cell line, the gastruloid model, or the organoid model, or a culture thereof in research on mechanisms of early embryonic development of humans.

A ninth objective of this patent is to provide use of the foregoing gastruloid stem cell line, the foregoing gastruloid model, the foregoing organoid model, or a tissue or organ derived from the cell line, the gastruloid model, or the organoid model, or a culture thereof in a diagnostic strategy and/or therapeutic strategy for a disease related to early embryonic development of humans.

A tenth objective of this patent is to provide use of the foregoing gastruloid stem cell line, the foregoing gastruloid model, the foregoing organoid model, or a tissue or organ derived from the cell line, the gastruloid model, or the organoid model, or a culture thereof in screening, validation, evaluation, assessment, or study of a medicament in terms of efficacy for prevention and/or treatment of a disease related to early embryonic development of humans.

The penicillin-streptomycin double antibiotic solution in the present invention contains 10000 units/mL penicillin and 10000 μg/mL streptomycin.

The ROCK inhibitor in the present invention includes but is not limited to Y-27632, ROCK-IN-1, and Chroman.

The gastruloid stem cell line CCRM-hGOSC-1 in the present invention is a new cell line obtained through in vitro induction from human embryonic stem cells, with characteristics similar to those of human gastrula stem cells. In a particular embodiment, the gastruloid model obtained on day 10 to day 20 after the gastruloid stem cell line CCRM-hGOSC-1 is injected into the testis of the nude mouse has characteristics of a human embryo during gastrulation, which can mimic gastrulation. A model similar to a human organ can be obtained on day 30 to day 90 after CCRM-hGOSC-1 is injected into the testis of the nude mouse, which can mimic organoid formation. The gastruloid stem cell line CCRM-hGOSC-1, the gastruloid model, the organoid model, or a tissue or organ derived from the cell line or the gastruloid model, or a culture thereof cannot develop into a human or animal body due to the lack of trophoblast and other types of cells.

The CCRM-hGOSC-1 cells provided in the present invention have at least the following characteristics:

Characteristic 1: The CCRM-hGOSC-1 cells proliferate rapidly, clones are formed visibly on day 3 after single-cell passage, the cells have relatively uniform sizes and shapes, which are round or oval, and the cell clones have clear boundaries.

Characteristic 2: The CCRM-hGOSC-1 cells exhibit active growth, good activity, high culture stability, and stable cell growth characteristics for in vitro culture.

Characteristic 3: The CCRM-hGOSC-1 cells are subjected to immunofluorescence staining through cell growing on a coverslip, and a cell population of the same clone expresses proteins of three germ layers: pluripotency genes OCT4 and SOX2, mesoderm genes EOMES and TBXT, and endoderm genes GATA4 and GATA6.

Characteristic 4: The CCRM-hGOSC-1 cells exhibit normal chromosome structures and quantities, and the cells have 44+XY chromosomes and belong to a diploid karyotype male cell line.

Characteristic 5: RNA sequencing shows that CCRM-hGOSC-1 cells express three germ layer protein markers, such as mesoderm genes MIXL1, EOMES, MESP1, WNT3, TBXT, and GSC, endoderm genes ELF3, FOXA2, CXCR4, GATA4, GATA6, and SOX17, and pluripotency-related markers POU5F1 (OCT4), NANOG, KLF4, and TFCP2L1. The proliferating cell line maintains the pluripotency of stem cells to a certain extent while exhibiting multilineage cell specialization, and exhibits gene expression of lineage cells from multiple germ layers, with characteristics similar to those of cells during gastrulation.

Characteristic 6: Human gastrulation can be mimicked on day 10 to day 20 after the CCRM-hGOSC-1 cells are injected into the testis of the nude mouse. On day 10 of sampling, it is observed that blastocyst-like bilaminar embryonic disc as well as amniotic cavity and yolk sac are formed in the testicular lumen. An amniotic organoid appears in OCT4 and SOX2 positive epiblast-like cell clusters. GATA6/GATA4/EOMES positive primitive endoderm-like cells migrate and assemble to form a primary yolk sac-like organoid. The epiblast and hypoblast are arranged in an orderly manner between the amniotic cavity and the yolk sac, forming an embryoid similar to embryos at CS5b and CS5c. In addition, it is observed that some embryoids start to develop toward gastruloids. Epiblast cells undergo epithelial-mesenchymal transition (EMT) to obtain EOMES/T positive and SOX2 negative gastrulation cells with reduced OCT4 expression. On day 20, gastrulation cells appear to form a gastruloid. OCT4 positive cells surround a cavity to further form an amniotic organoid, and gradually differentiate on the apical side of the amniotic organoid into KRT7/GATA2/GATA3 positive amniotic-like epithelial cells. In this case, EOMES/T positive gastrulation cells appear, and yolk sacs of some embryos are gradually covered by proliferating and migrating mesoendodermal cells.

Characteristic 7: Organoid formation can be mimicked on day 30 to day 90 after CCRM-hGOSC-1 is injected into the testis of the nude mouse. On day 30 to day 40, the amniotic cavity proliferates and enlarges, and neural ectoderm appears. On day 50 to day 90, hGOSCs differentiate to form a tissue or organ from ectoderm, mesoderm, and endoderm, such as neuroepithelium, smooth muscle, or intestines. Endoderm: Through immunofluorescence CDX2&GATA6 labeling and HE staining morphology analysis, it can be learned that injected hGOSCs gradually form an intestinal structure with time, and form a muscle-coated intestinal organoid on day 90. Mesoderm: Through immunofluorescence SOX9 labeling cartilage and ACTA2 labeling muscle, with HE staining morphology analysis, it can be learned that hGOSCs form muscle and cartilage 90 days after injection. Ectoderm: 30 days after injection of hGOSCs into the testicular lumen, a small number of primordial germ cells are found through SOX17, BLIMP1, and TFAP2C labeling, and amniotic epithelial cells are found in the vicinity of the primordial germ cells through GATA2, GATA3, and KRT7 labeling. With HE staining morphology, as well as KER15 indicating keratinocytes and ACTA2 indicating muscle, the skin structure is found. HE and immunofluorescence staining indicates that neuroepithelial cells with stemness appear on day 40 to day 50, and the neuroepithelial cells differentiate and form neurons on day 70 to day 90, where OTX2 and SOX2 label the neuroepithelial or radial glial cells, and TUJ1 and DCX label the neurons.

The gastruloid stem cell line DYR0100-hGOSC-1 in the present invention is a new cell line obtained through in vitro induction from pluripotent stem cells, with characteristics similar to those of gastrula stem cells. In a particular embodiment, the gastruloid model obtained through differentiation of the gastruloid stem cell line DYR0100-hGOSC-1 has characteristics of a human embryo during gastrulation, which can mimic gastrulation for studying morphologic developmental characteristics and genetic functions during gastrulation. The gastruloid stem cell line DYR0100-hGOSC-1, the gastruloid model, the organoid model, or a tissue or organ derived from the cell line, the gastruloid model, or the organoid model, or a culture thereof cannot develop into a body due to the lack of trophoblast and other types of cells.

DYR0100-hGOSC-1 provided in the present invention has at least the following characteristics:

Characteristic 1: DYR0100-hGOSC-1 proliferates rapidly, clones are formed visibly on day 3 after single-cell passage, the cells have relatively uniform sizes and shapes, which are round or oval, and the cell clones have clear boundaries.

Characteristic 2: DYR0100-hGOSC-1 exhibits active growth, good activity, high culture stability, and stable cell growth characteristics for in vitro culture.

Characteristic 3: DYR0100-hGOSC-1 is subjected to immunofluorescence staining through cell growing on a coverslip, and a cell population of the same clone expresses proteins of three germ layers: pluripotency genes OCT4 and SOX2, mesoderm genes EOMES, TBXT, CDX2, and MIXL1, and endoderm genes GATA4, GATA6, SOX17, and OTX2.

Characteristic 4: DYR0100-hGOSC-1 exhibits normal chromosome structures and quantities, and the cells have 44+XY chromosomes and belong to a diploid karyotype male cell line.

Characteristic 5: DYR0100-hGOSC-1 maintains the pluripotency of stem cells to a certain extent while exhibiting multilineage cell specialization, and exhibits gene expression of lineage cells from multiple germ layers, with characteristics similar to those of cells during gastrulation. RNA sequencing shows that DYR0100-hGOSC-1 expresses three germ layer protein markers, such as mesoderm genes MIXL1, EOMES, MESP1, WNT3, TBXT, and GSC, endoderm genes ELF3, FOXA2, CXCR4, GATA4, GATA6, and SOX17, and pluripotency-related markers POU5F1 (OCT4), NANOG, KLF4, TFCP2L1, and SOX2.

Characteristic 6: DYR0100-hGOSC-1 can form spheres within 12 hours, and exhibit good cell activity, high induction stability, and stable cell growth characteristics for four continuous days of gastruloid culture.

Characteristic 7: On day 0 and day 1 of gastruloid induction, the gastruloid stem cells start to assemble and form a stable three-dimensional structure, inside which epiblast (expressing OCT4 and SOX2), primitive streak (expressing TBXT and MIXL1), and endoderm (expressing SOX17, OTX2, and FOXA2) cells appear, and a small number of amniotic epithelial cells (expressing CDX2) and extraembryonic mesoderm cells (expressing LUM) also appear.

Characteristic 8: On day 2 to day 4 of gastruloid induction, lineage separation of epiblast (expressing OCT4 and SOX2), mesoderm (expressing EOMES and MESP1) developed from the primitive streak, and endoderm (expressing SOX17, OTX2, and FOXA2) starts to occur inside the three-dimensional model formed through the assembly of the gastruloid stem cells, and OCT4 and SOX2 positive epiblast cells form an amniotic organoid.

Characteristic 9: The gastruloid after four days of induction culture is similar to an embryo at Carnegie stage 7.

### Beneficial Effects

According to the present invention, the method for constructing the gastruloid stem cell line, the gastruloid stem cell line obtained by using the method, and the construction of the model derived from the cell line provide a platform for in vitro research on human gastrulation, to understand research on the complexity of early embryonic development of humans and provide a research platform for developing clinical therapies for diseases related to early peri-implantation embryos.

### BRIEF DESCRIPTION OF THE DRAWINGS

FIG. 1 shows cell morphology according to Example 2 of the present invention, where FIG. 1A shows a human embryonic stem cell, FIG. 1B shows a nascent mesoderm-like cell, FIG. 1C shows a primordial germ cell-like cell sphere, and FIG. 1D shows a gastruloid stem cell.

FIG. 2 shows a growth curve according to Example 2 of the present invention.

FIG. 3 shows results of immunofluorescence identification according to Example 2 of the present invention, where OCT4 and SOX2 are pluripotency genes, EOMES and TBXT are mesoderm genes, and GATA4 and GATA6 are endoderm genes, at a scale of 100 μm.

FIG. 4 shows results of karyotyping according to Example 2 of the present invention.

FIG. 5 shows results of RNA sequencing according to Example 2 of the present invention.

FIG. 6 shows results of in vivo gastruloid HE according to Example 3 of the present invention, where the asterisk * indicates the amniotic cavity, the wide arrow [symbol] points to the amniotic epithelial cells, the square ▪ indicates the yolk sac, the narrow arrow ↑ points to the epiblast cells, the triangle ▴ points to the primitive endoderm cells, and the five-pointed star ★ indicates the gastrulation cells, at a scale of 100 μm.

FIG. 7 shows results of in vivo gastruloid IF according to Example 3 of the present invention, where OCT4/SOX2 labels epiblast cells, GATA6/GATA4 labels primitive endoderm cells, T+EOMES labels gastrulation cells, and KRT7/GATA2/GATA3 labels amniotic epithelial cells, at a scale of 20 μm.

FIG. 8 shows results of in vivo organoid HE according to Example 4 of the present invention, where FIG. 8A shows a digestive tract organoid from endoderm, FIG. 8B shows a skin organoid from ectoderm, and FIG. 8C shows a cartilaginous organoid from mesoderm.

FIG. 9 shows results of in vivo organoid IF according to Example 4 of the present invention, where GATA6 and CDX2 label the digestive tract, KRT15 and ACTA2 label the skin, and SOX9 labels the cartilage.

FIG. 10 shows cell morphology at different passages P1 (FIG. 10A), P7 (FIG. 10B), P20 (FIG. 10C), and P30 (FIG. 10D) according to Example 6 of the present invention, at a scale of 100 μm.

FIG. 11 shows a growth curve according to Example 6 of the present invention.

FIG. 12 shows results of immunofluorescence identification according to Example 6 of the present invention, at a scale of 100 μm.

FIG. 13 shows results of karyotyping according to Example 6 of the present invention.

FIG. 14 shows results of RNA sequencing of a gastruloid stem cell line according to Example 6 of the present invention.

FIG. 15 shows images under white light of gastruloids on day 0 to day 4 of induction culture according to Example 9 of the present invention, at a scale of 100 μm.

FIG. 16 shows images under white light of sampling of gastruloids on day 0 to day 4 of induction culture according to Example 9 of the present invention, at a scale of 100 μm.

FIG. 17 shows a growth curve of 1-4 days of gastruloid induction according to Example 9 of the present invention.

FIG. 18 shows results of IF of assembling gastruloid stem cells into a three-dimensional structure on day 1 of gastruloid induction according to Example 9 of the present invention, at a scale of 50 μm.

FIG. 19 shows results of IF of proamniotic cavity formation and mesoendodermal lineage specialization after gastruloid model induction is completed on day 2 to day 4 of gastruloid induction according to Example 9 of the present invention, at a scale of 50 μm.

FIG. 20 shows results of RNA sequencing of a gastruloid model according to Example 9 of the present invention.

### BIOLOGICAL MATERIAL DEPOSITION INFORMATION

The gastruloid stem cell line with an accession number of CCTCC NO. C2022114 has been deposited in the China Center for Type Culture Collection, deposition date: Apr. 27, 2020, deposition address: Wuhan University, Wuhan, taxonomic name: human gastruloid stem cell line CCRM-hGOSC-1.

The gastruloid stem cell line with an accession number of CCTCC NO. C2022115 has been deposited in the China Center for Type Culture Collection, deposition date: Apr. 27, 2020, deposition address: Wuhan University, Wuhan, taxonomic name: human gastruloid stem cell line DYR0100-hGOSC-1.

### DETAILED DESCRIPTION

### 1. Method for Constructing a Gastruloid Stem Cell Line by Inducing Human Embryonic Stem Cells

The present invention discloses a method for inducing human embryonic stem cells into the gastruloid stem cell line, and constructs a human in vitro post-implantation gastruloid model derived from the cell line. The reagents, instruments, and cell lines used in the present invention are commercially available.

The human embryonic stem cells are provided by the Center of Reproductive Medicine of Jiangsu Province Hospital, with the cell name of CCRM-hESCs-22 (46, XY).

(1) Sources of Components in the Medium Used for Inducing the Human Embryonic Stem Cells into the Human Gastruloid Stem Cell Line In Vitro:
GMEM (Glasgow's MEM) medium: 11710035, purchased from Gibco, USA;
serum replacement (Thermo Fisher confidential formula): 10828028, purchased from Gibco, USA;
fetal bovine serum: 12483020, purchased from Gibco, USA;
Gibco® MEM non-essential amino acids: 11140076, purchased from Gibco, USA;
GlutaMAX™ additive: 35050061, purchased from Gibco, USA;
sodium pyruvate additive: 11360070, purchased from Gibco, USA;
penicillin-streptomycin double antibiotic solution: 15140122, Gibco, USA;
β-mercaptoethanol: 21985023, purchased from Gibco, USA;
recombinant human bone morphogenetic protein 4:314-BP, purchased from R&D Systems, USA;
recombinant human stem cell factor: 7734-LF, purchased from R&D Systems, USA;
recombinant human leukemia inhibitory factor: 225-SC, purchased from R&D Systems, USA;
recombinant human epidermal growth factor: 236-EG, purchased from R&D Systems, USA;
recombinant human activin A protein: 338-AC, purchased from R&D Systems, USA;
glycogen synthase kinase-3 (GSK-3) inhibitor: specifically CHIR 99021;
ROCK inhibitor: specifically Y-27632, HY-10071, purchased from MCE, USA;
adenylate cyclase activator: specifically forskolin, 1099, purchased from R&D Systems, USA; and
PDE4 inhibitor: specifically Rolipram, 0905, purchased from R&D Systems, USA.
   (2) Sources of Components in the Medium Used for Inducing the Human Pluripotent Stem Cells into the Post-Implantation Blastoid In Vitro:
mTeSR™ 1 medium: #85850, purchased from STEMCELL Technologies, Canada;
Essential 6 medium: A1516401, purchased from Gibco, USA;
recombinant human fibroblast growth factor 2:3718-FB, purchased from R&D Systems, USA;
recombinant human Noggin protein: HY-P7051A, purchased from MCE, USA;
ROCK inhibitor: specifically Y-27632, HY-10071, purchased from MCE, USA; and
WNT inhibitor: specifically IWP-2, S7085, purchased from Selleck, USA.
   (3) Preparation of a GK15-1 Medium Containing the ROCK Inhibitor Y-27632 in Induction from Embryonic Stem Cells to Mesoderm (Stage 1):

81% of basal medium GMEM in percentage by volume, 15% of serum replacement KOSR in percentage by volume, 1% of penicillin-streptomycin double antibiotic solution (containing 10000 units/mL penicillin and 10000 μg/mL streptomycin) in percentage by volume, 1% of 10 mM non-essential amino acids in percentage by volume, 1% of 200 mM GlutaMAX additive in percentage by volume, 1% of 100 mM sodium pyruvate additive in percentage by volume, 0.1 mM β-mercaptoethanol, 50 ng/mL recombinant human activin A, 3 μM glycogen synthase kinase-3 (GSK-3) inhibitor CHIR 99021, and 10 μM ROCK inhibitor Y-27632.

(4) Preparation of a GK15-1 Medium in Induction from Embryonic Stem Cells to Mesoderm (Stage 1):

81% of basal medium GMEM in percentage by volume, 15% of serum replacement KOSR in percentage by volume, 1% of penicillin-streptomycin double antibiotic solution (containing 10000 units/mL penicillin and 10000 μg/mL streptomycin) in percentage by volume, 1% of 10 mM non-essential amino acids in percentage by volume, 1% of 200 mM GlutaMAX additive in percentage by volume, 1% of 100 mM sodium pyruvate additive in percentage by volume, 0.1 mM β-mercaptoethanol, and multiple cytokines: 50 ng/ml recombinant human activin A and 3 μM glycogen synthase kinase-3 (GSK-3) inhibitor CHIR 99021.

(5) Preparation of a GK15-2 Medium Containing the ROCK Inhibitor Y-27632 in Induction from Nascent Mesoderm-Like Cells to Primordial Germ Cell-Like Cells (Stage 2) and in Cell Purification:

81% of basal medium GMEM in percentage by volume, 10% of serum replacement KOSR in percentage by volume, 1% of penicillin-streptomycin double antibiotic solution (containing 10000 units/mL penicillin and 10000 μg/mL streptomycin) in percentage by volume, 1% of 10 mM non-essential amino acids in percentage by volume, 1% of 200 mM GlutaMAX additive in percentage by volume, 1% of 100 mM sodium pyruvate additive in percentage by volume, 0.1 mM β-mercaptoethanol, 100 ng/mL recombinant human stem cell factor, 200 ng/mL recombinant human bone morphogenetic protein 4, 1000 U/mL recombinant human leukemia inhibitory factor, 50 ng/mL recombinant human epidermal growth factor, and 10 μM ROCK inhibitor Y-27632.

(6) Preparation of a GK15-2 Medium in Induction from Nascent Mesoderm-Like Cells to Primordial Germ Cell-Like Cells (Stage 2) and in Cell Purification:

81% of basal medium GMEM in percentage by volume, 15% of serum replacement KOSR in percentage by volume, 1% of penicillin-streptomycin double antibiotic solution (containing 10000 units/mL penicillin and 10000 μg/mL streptomycin) in percentage by volume, 1% of 10 mM non-essential amino acids in percentage by volume, 1% of 200 mM GlutaMAX additive in percentage by volume, 1% of 100 mM sodium pyruvate additive in percentage by volume, 0.1 mM β-mercaptoethanol, 200 ng/mL recombinant human bone morphogenetic protein 4, 100 ng/ml recombinant human stem cell factor, 1000 U/mL recombinant human leukemia inhibitory factor, and 50 ng/mL recombinant human epidermal growth factor.

### (7) Preparation of a GK10 Medium Containing the ROCK Inhibitor Y-27632 in the In Vitro Expansion of a Gastruloid Stem Cell Line:

83.5% of basal medium GMEM in percentage by volume, 10% of serum replacement KOSR in percentage by volume, 2.5% of fetal bovine serum FBS in percentage by volume, 1% of penicillin-streptomycin double antibiotic solution (containing 10000 units/mL penicillin and 10000 μg/mL streptomycin) in percentage by volume, 1% of 10 mM non-essential amino acids in percentage by volume, 1% of 200 mM GlutaMAX additive in percentage by volume, 1% of 100 mM sodium pyruvate additive in percentage by volume, 0.1 mM β-mercaptoethanol, 10 μM forskolin, 10 μM Rolipram, 100 ng/mL recombinant human stem cell factor, and 10 μM ROCK inhibitor Y-27632.

### (8) Preparation of a GK10 Medium in the In Vitro Expansion of a Gastruloid Stem Cell Line:

83.5% of basal medium GMEM in percentage by volume, 10% of serum replacement KOSR in percentage by volume, 2.5% of fetal bovine serum FBS in percentage by volume, 1% of penicillin-streptomycin double antibiotic solution (containing 10000 units/mL penicillin and 10000 μg/mL streptomycin) in percentage by volume, 1% of 10 mM non-essential amino acids in percentage by volume, 1% of 200 mM GlutaMAX additive in percentage by volume, 1% of 100 mM sodium pyruvate additive in percentage by volume, 0.1 mM β-mercaptoethanol, 10 μM forskolin, 10 μM Rolipram, and 100 ng/ml recombinant human stem cell factor.

### Example 1: Construction of the Gastruloid Stem Cell Line CCRM-hGOSC-1

This example of the present invention provides gastruloid stem cells named human gastruloid stem cell line CCRM-hGOSC-1. The cell line is deposited in the China Center for Type Culture Collection with an accession number of CCTCC NO. C2022114.

(1) Induction from Embryonic Stem Cells to Mesoderm (Stage 1):

(1-1) The human embryonic stem cells were CCRM-hESCs-22 (46, XY) provided by the Center of Reproductive Medicine of Jiangsu Province Hospital. The human embryonic stem cells were digested into single cells with TrypLE Select when the cells grew to 80%-90% confluency, and centrifuged by using a horizontal centrifuge at 1300 rpm for 3 min. Then, the cells were resuspended with PBS to obtain a cell suspension 1.

(1-2) The cell suspension 1 was centrifuged, a supernatant was removed, and a GK15-1 medium containing Y-27632 was added to resuspend cells, to obtain a cell suspension 2. Each 1 mL of the GK15-1 medium containing Y-27632 contained 1×10⁶ cells.

(1-3) The cell suspension 2 was inoculated into a six-well plate pre-coated with matrigel in a density of 8×10⁵ cells/cm². The cells in the six-well plate were shaken uniformly and then transferred to an incubator at 37° C. under 5% CO₂ for culture.

(1-4) On day 2 of the culture, the spent medium was replaced with a GK15-1 medium. The medium was changed daily until nascent mesoderm-like cells were obtained.

The foregoing steps of induction from the embryonic stem cells to mesoderm were implemented on day 0 to day 2 of the construction of the gastruloid stem cell line.

(2) Induction from the Nascent Mesoderm-Like Cells to Primordial Germ Cell-Like Cells (Stage 2):

(2-1) The nascent mesoderm-like cells obtained in (1-4) were digested into single cells with TrypLE Select when the cells grew to 80%-90% confluency, and centrifuged by using a horizontal centrifuge at 1300 rpm for 3 min. Then, the cells were resuspended with PBS to obtain a cell suspension 3.

(2-2) The cell suspension 3 was centrifuged, a supernatant was removed, and a GK15-2 medium containing Y-27632 was added to further resuspend cells, to obtain a cell suspension 4. Each 1 mL of the GK15-2 medium containing Y-27632 contained 1×10⁵ cells.

(2-3) The cell suspension 4 obtained in (2-2) was re-inoculated into a transparent low-attachment 96-well plate with round bottoms for sphere-forming culture with 5×10³ cells per well. On day 2 of the culture, the spent medium was replaced with a GK15-2 medium. The medium was changed daily until cell spheres containing primordial germ cell-like cells were obtained.

The foregoing steps of induction from the nascent mesoderm-like cells to endoderm were implemented on day 3 to day 6 of the construction of the gastruloid stem cell line.

### (3) Cell Purification:

(3-1) The cell spheres obtained in (2-3) were digested into single cells with collagenase IV and 0.25% Trypsin-EDTA trypsin, and centrifuged by using a horizontal centrifuge at 1300 rpm for 3 min. The cells were resuspended with a GK15-2 medium to obtain a cell suspension 5.

(3-2) CD326 and CD49f double-positive cells were separated by using a flow cytometer from the cell suspension 5 obtained in (3-1), and centrifuged at 200 g for 3 min. A GK10 medium containing Y-27632 was added to resuspend cells, to obtain a cell suspension 6. Each 1 mL of the GK10 medium containing Y-27632 contained 1×10⁵ cells.

### (4) Cell Expansion:

Mouse embryo fibroblasts treated with mitomycin C were thawed one day in advance and spread in a well plate as feeder cells. The cell suspension 6 obtained in (3-2) was inoculated in a density of 2×10⁴ cells/cm² onto the feeder cells prepared in advance, shaken uniformly, and placed in an incubator at 37° C. under 5% CO₂. The medium was replaced with a fresh GK10 medium after 24 hours. The medium was changed daily. The gastruloid stem cell line CCRM-hGOSC-1 was obtained when formed clones can be observed, which was deposited in the China Center for Type Culture Collection with an accession number of CCTCC NO. C2022114.

### Example 2: Description of Biological Characteristics of Cells

### 1.1 Cell Morphology Observation

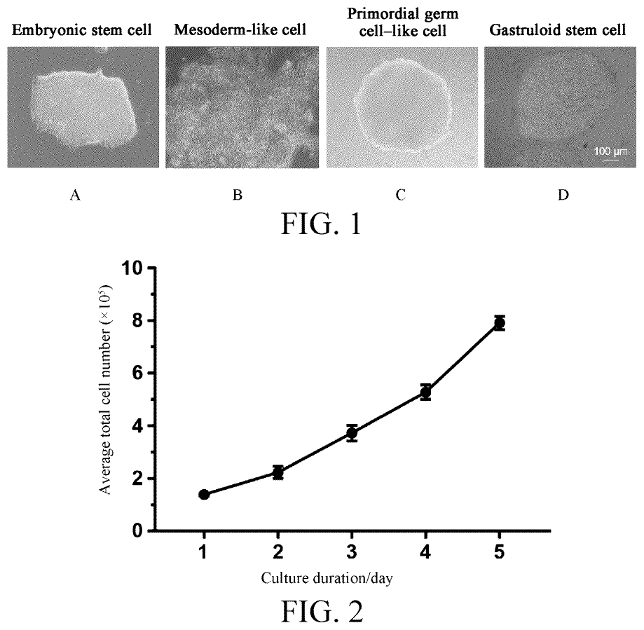

The human gastruloid stem cells CCRM-hGOSC-1 were observed under an inverted microscope. The cells proliferate rapidly, clones are formed visibly on day 2 after single-cell passage, the cells have relatively uniform sizes and shapes, which are round or oval, and the cell clones have clear boundaries. The morphology observation results are shown in FIG. 1.

### 1.2 Growth Curve Determination

### 1.2.1 Steps of Growth Curve Determination

When the confluency of the human gastruloid stem cells CCRM-hGOSC-1 reached 70%-90%, the medium was removed, the cells were washed with PBS (0.01 M, pH 7.4) at least twice to remove the spent medium and detached cells in poor condition, digested with TrypLE Select, and observed under a microscope. The digested cells were collected during digestion. A GK10 medium was added to the collected cells to stop the digestion. Finally, all the cells were digested. The cells were centrifuged at 1000 rpm for 5 min. A supernatant was removed. A GK10 medium was added. Each 1 mL of the GK10 medium contained 1×10⁵ cells.

The cells were inoculated in a density of 1×10⁵ cells per well (of a 12-well plate) onto the feeder cells prepared in advance, shaken uniformly, and placed in an incubator at 37° C. under 5% CO₂. The medium was replaced with a fresh GK10 medium after 24 hours. The medium was changed daily. From the time of inoculation, the medium was removed from three wells every 24 hours, TrypLE Select was added for digestion, the cells were suspended, and total cell numbers in the three wells were averaged, where the cells in each well were counted three times and averaged, while the cells in other wells were still cultured, until day 5. The medium was changed daily.

### 1.2.2 Results of Growth Curve Determination

The determination was carried out for five consecutive days to obtain growth curve data shown in Table 1 below.

Table 1 shows the growth curve data obtained through five consecutive days of determination.

TABLE 1

| Culture duration (day) | Average total cell number |
|---|---|
| 1 | 1.35 × 10⁵ |
| 2 | 2.23 × 10⁵ |
| 3 | 3.73 × 10⁵ |
| 4 | 5.28 × 10⁵ |
| 5 | 7.91 × 10⁵ |

A cell growth curve shown in FIG. 2 is obtained based on the cell growth curve data in Table 1. In FIG. 2, horizontal coordinates indicate the culture duration (day), and vertical coordinates indicate the cell number (×105).

With reference to the growth curve data in Table 1 and the growth curve shown in FIG. 2, it can be learned that the CCRM-hGOSC-1 cells grew well for the five consecutive days.

In conclusion, the CCRM-hGOSC-1 cells proliferate rapidly, and exhibit active growth, good activity, high culture stability, and stable cell growth characteristics for in vitro culture.

### 1.3 Immunofluorescence Identification

### 1.3.1 Steps of Immunofluorescence Identification

A small round coverslip was sterilized with 75% ethanol and by ultraviolet light, and then transferred into a cell culture dish. The coverslip was coated with Fibronectin to improve attachment. Then, the CCRM-hGOSC-1 cells were inoculated and cultured according to steps of normal cell passage culture.

Before passage of the cultured CCRM-hGOSC-1 cells, the small round coverslip was taken out and placed in a dish for fixation with 4% PFA for 40 min.

The 4% PFA fixation solution was removed. The cells were washed with PBS three times. 5% BSA was added for blocking at room temperature for 2 h.

The 5% BSA blocking buffer was removed. The cells were incubated at 4° C. overnight with an antibody diluted proportionally.

The primary antibody was removed. The cells were washed with PBS three times. The cells were incubated at room temperature for 2 h with diluted secondary antibody and live cell staining solution Hoechst 33342 that were added to the dish.

The secondary antibody was removed. The cells were washed with PBS three times. Glycerol was added onto a slide. The small round coverslip was taken out of the dish and inverted onto the slide with glycerol added, and then fixed with nail polish. A confocal fluorescence microscope was used for imaging.

### 1.3.2 Results of Immunofluorescence Identification

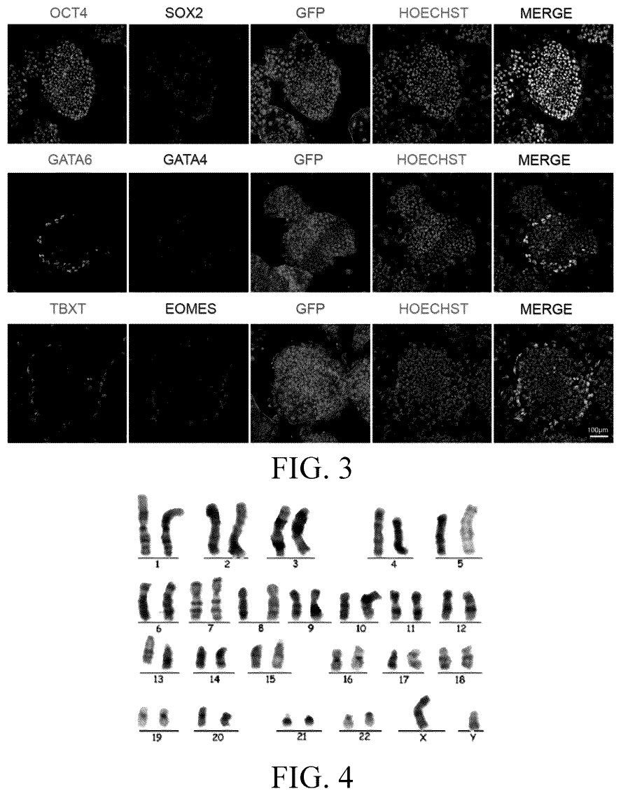

The results of immunofluorescence identification are shown in FIG. 3. The proliferating cell line was subjected to immunofluorescence staining through cell growing on a coverslip, to identify genes in each germ layer. It is found that pluripotency genes OCT4 and SOX2, mesoderm genes EOMES and TBXT, and endoderm genes GATA4 and GATA6 are partially expressed in cell clones.

### 1.4 Karyotyping Identification

### 1.4.1. Steps of Karyotyping Identification

### Cell Culture

When the confluency of the cells reached 70%-90%, the medium was removed, the cells were washed with PBS (0.01 M, pH 7.4) at least twice to remove the spent medium and detached cells in poor condition, digested with 1-2 mL of TrypLE Select, and observed under a microscope. The digested cells were collected during digestion. A GK10 medium was added to the collected cells to stop the digestion. Finally, all the cells were digested. The cells were centrifuged at 1000 rpm for 5 min. A supernatant was removed.

Treatment with Colchicine

20 μg/mL colchicine was added to the cell medium at 1:200 to reach a final concentration of 0.1 μg/mL. Then, the cells were incubated in an incubator at 37° C. for 3 h to obtain colchicine-treated gastruloid stem cells CCRM-hGOSC-1.

### Hypotonic Treatment

A 0.56% KCl hypotonic solution was pre-warmed to 37° C. The culture dish was taken out, and the colchicine-treated gastruloid stem cells CCRM-hGOSC-1 therein were digested into a single-cell suspension. The cell suspension was transferred to a 15 mL centrifuge tube and centrifuged at 2000 rpm for 10 min. A supernatant was removed. To the cell pellet was added 9 mL of the 0.56% KCl hypotonic solution pre-warmed to 37° C. The cell pellet was pipetted gently using a glass dropper with a rubber bulb for about 50 times, to carry out hypotonic treatment at 37° C. for 40 min.

### Fixation

A fixation solution (methanol:glacial acetic acid=4:1) was prepared and mixed uniformly at room temperature. The cells with 1 mL of the fixation solution added were pipetted gently and then centrifuged at 300 g for 10 min. A supernatant was removed. The cells with 10 mL of fresh fixation solution added were pipetted gently into a single-cell suspension to carry out fixation at room temperature for 1 h, and then centrifuged at 300 g for 10 min. A supernatant was removed. The cells with 10 mL of fresh fixation solution added were pipetted gently and resuspended to carry out fixation at room temperature for 30 min, and then centrifuged at 300 g for 10 min. A supernatant was removed. A small amount (0.2-0.6 mL) of fixation solution was added based on the amount of cells precipitated to resuspend the cells.

### Preparation of Slides

An alcohol lamp was lighted. A clean slide was taken out of distilled water without drying, and placed obliquely on a waste tank, while the cell suspension 30-60 cm above the slide was dropped on the slide using a pipette. One drop of the cell suspension was placed at each of three different positions of each slide, followed immediately by cauterizing the slide on the back by the alcohol lamp five times. The slide was marked and transferred to an oven at 37° C. for overnight baking.

### Staining

The slide with the human gastruloid stem cell sample was taken out of the oven at 37° C. and placed in an oven at 80° C. for 2.5 h, followed by banding. 0.25% Trypsin-EDTA was pre-warmed to 37° C. for use. The slide was immersed in 0.25% Trypsin-EDTA for 30-40s, and then taken out and rinsed on both sides under running water two or three times. The slide was immersed in Giemsa stain pre-warmed to 37° C. for staining for about 10 min, then rinsed on both sides under running water two or three times, and wiped using lens wiping paper until the surface was dried.

### Microscopic Examination

Well-dispersed division phases of moderate lengths were selected under a low-power microscope, and then observed using a lens immersed in oil and imaged, to obtain results of karyotyping identification.

### 1.4.2 Results of Karyotyping Identification

The results of karyotyping identification are shown in FIG. 4. With reference to FIG. 4, through the karyotyping identification, it can be learned that the cells exhibit normal chromosome structures and quantities, and the cells have 44+XY chromosomes and belong to a diploid karyotype male cell line.

### 1.5 RNA Sequencing Identification 1.5.1 Steps of RNA Sequencing Identification

When the confluency of the CCRM-hGOSC-1 cells reached 70%-90%, the medium was removed, and the cells were washed with PBS (0.01 M, pH 7.4) at least three times to remove the spent medium and detached cells in poor condition. The CCRM-hGOSC-1 cells were digested with TrypLE Select into single cells, and then collected. A GK10 medium was added to stop the digestion. The cells were centrifuged at 1000 rpm for 5 min. A supernatant was removed. Then, the cells with 1 mL of Trizol added were stored in a cold (−80° C.) refrigerator.

RNA was extracted from the CCRM-hGOSC-1 cell sample by the phenol-chloroform method.

The cell sample was taken out of the cold refrigerator and thawed on ice, followed by adding 200 μL of chloroform, shaken vigorously and then left to stand at room temperature for 3 min, and then centrifuged at 12000 rpm for 15 min using a benchtop high-speed centrifuge pre-cooled to 4° C.

The centrifuged sample was transferred on ice, and a supernatant was taken. To the supernatant, isopropanol was added at 1:1, and hepatic glycogen was added at 200:1, shaking uniformly. Then, the mixture was stored in a cold (−80° C.) refrigerator for 30 min, then taken out and thawed on ice, and then centrifuged at 12000 rpm for 15 min using a benchtop high-speed centrifuge pre-cooled to 4° C.

A supernatant was removed. The cell pellet was washed once with enzyme-free 75% alcohol, and centrifuged at 12000 rpm for 10 min using a benchtop high-speed centrifuge pre-cooled to 4° C. A supernatant was removed. The cells were dried in a fume hood to remove the remaining 75% alcohol.

The dried cell sample was re-dissolved in 10-20 μL of enzyme-free water based on the amount of RNA, and then transferred into an enzyme-free EP tube for sequencing.

### 1.5.2 Results of RNA Sequencing Identification

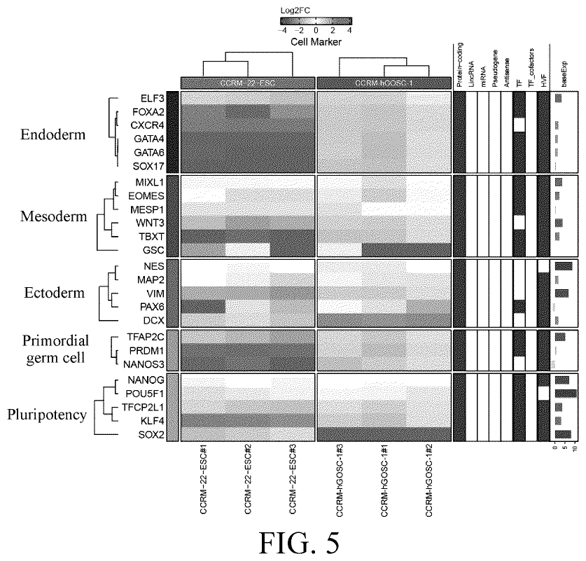

The results of RNA sequencing identification are shown in FIG. 5. Through the comparison in transcriptome sequencing between the CCRM-hGOSC-1 proliferating cells and the human embryonic stem cells (hESCs), it can be found that, compared with the hESCs, the proliferating cells exhibit expression of elevated transcript levels of marker genes of multilineage cells (ectoderm, endoderm, mesoderm, amnion, and primordial germ cells). Specifically, the expression of the mesoderm genes MIXL1, EOMES, MESP1, WNT3, TBXT, and GSC and the expression of the endoderm genes ELF3, FOXA2, CXCR4, GATA4, GATA6, and SOX17 greatly increase, although the expression of SOX2 decreases, POU5F1 (OCT4) and NANOG are still expressed, and the expression of naïve pluripotency factors KLF4 and TFCP2L1 increases, indicating that the proliferating cell line still exhibits pluripotency. The proliferating cell line maintains the pluripotency of stem cells to a certain extent while exhibiting multilineage cell specialization, and exhibits gene expression of lineage cells from multiple germ layers, with characteristics similar to those of cells during gastrulation.

### Example 3: Formation of a Gastruloid Model by Injecting the Cell Line into a Mouse

The CCRM-hGOSC-1 cells have characteristics during gastrulation, which can mimic gastrulation for studying morphologic developmental characteristics and genetic functions during gastrulation. The gastruloid can be formed 10-20 days after the cells are injected into the testis of the mouse.

1. Injection into the Testis of the Nude Mouse

Preparation: A suitable capillary glass needle was made using a needle puller with parameters: Heat 515, Pull 100, Trip 75, delay 75.

When the confluency of the CCRM-hGOSC-1 cells reached 70%-90%, the medium was removed, the cells were washed with PBS (0.01 M, pH 7.4) at least twice to remove the spent medium and detached cells in poor condition, digested with 1-2 mL of TrypLE, and observed under a microscope. The digested cells were collected during digestion. A GK10 medium was added to the collected cells to stop the digestion. Finally, all the cells were digested. The cells were centrifuged at 1000 rpm for 5 min. A supernatant was removed.

The cells were resuspended with a GK10 medium in a density of 2×10⁶ cells/mL. 5-6×10⁵ cells were injected into one testis of the BALB/c Nude mouse each time.

The mouse after injection was fed normally.

### 2. Validation of Gastrulation

### 2.1 Testicular Tissue Sampling and Preparation

The testicular tissue of the mouse after injection in 1 was sampled on day 10 and day 20 after the injection.

The sampled testicular tissue was placed in 4% PFA or mDF fixation solution for fixation at room temperature for 6 h, and then cut in half.

The fixation solution was removed after 42 hours. The testicular tissue was dehydrated at room temperature. Specifically, the testicular tissue was dehydrated with 70% alcohol for 24 h, dehydrated with 80% alcohol for 2 h, dehydrated with 90% alcohol for 2 h, dehydrated with 100% alcohol for 1 h, dehydrated with alcohol and xylene in 1:1 for 25 min, and cleared with xylene for 25 min.

The tissue block was transferred into an embedding cassette and infiltrated into paraffin (1) and paraffin (2) each for 45 min.

The embedded tissue was cut into consecutive 5 μm-thick sections. The paraffin sections were floated on a water bath to stretch the paraffin sections. Then, an intact tissue section was selected and lifted to adhere to a slide.

The slide was dried at 65° C. overnight. The slide was transferred to a 37° C. oven for 30 min and then stored at room temperature for a long time.

### 2.2 HE (Hematoxylin and Eosin) Staining

The paraffin section was deparaffinized and incubated in xylene (1) and xylene (2) each at 37° C. for 15 min.

The tissue was rehydrated in alcohol at room temperature in a gradient: 100% alcohol (1), 100% alcohol (2), 90% alcohol, 80% alcohol, and 70% alcohol, each for 2 min, and finally immersed in tap water for 10 min.

The section was stained in hematoxylin for 40 s and then rinsed under running water for 5 min.

The section was washed once with 1% HCl, and rinsed under running water for 10 min.

The section was stained in eosin for 3 min.

After staining, the tissue section was dehydrated in alcohol in a gradient: 70% alcohol, 80% alcohol, 90% alcohol, 100% alcohol (1), and 100% alcohol (2), each for 2 min, and in xylene (1) and xylene (2) each for 15 min.

The tissue on which resin was dropped was covered by a coverslip for cover slipping, placed in an oven at 37° C. for 2 h, then taken out and stored at room temperature, and then placed under an upright microscope for imaging.

### 2.3 Immunofluorescence (IF) Staining

The tissue section was deparaffinized, rehydrated, and transferred in tap water, which were the same as in HE staining.

Antigen retrieval: 200 mL of acidic antigen retrieval solution was prepared and added into an antigen retrieval box. The section was transferred into the retrieval box and then placed in a microwave at high power for 3 min and at low power for 7 min for antigen retrieval, and then naturally cooled down to room temperature.

Blocking: The slide was washed with PBS. A circle was drawn around the tissue using an immunohistochemical pen, and 100 μL of 5% BSA solution was added within the circle for incubation at room temperature for 2 h.

Incubation with primary antibody: The liquid on the tissue was aspirated as much as possible. The tissue was then covered by the antibody diluted proportionally with 5% BSA and incubated at 4° C. overnight.

The tissue with the antibody removed was washed with PBS three times, for 5 min each time.

Fluorescent secondary antibody and Hoechst 33342 were diluted with 5% BSA at 1:1000, and added to the tissue slide for incubation at room temperature for 2 h. The slide was washed with PBS three times, for 5 min each time.

The slide was cover-slipped with glycerol. A confocal fluorescence microscope was used for imaging.

### 2.4 Results of Gastrula HE and IF Validation

After the gastruloid stem cell line CCRM-hGOSC-1 in the present invention is injected into the testis of the mouse, on day 10 of sampling, it can be seen that blastocyst-like bilaminar embryonic disc as well as amniotic cavity and yolk sac are formed in the lumen. A cavity similar to the proamniotic cavity appears in OCT4 and SOX2 positive epiblast-like cell clusters. GATA6/GATA4/EOMES positive primitive endoderm-like cells migrate and assemble to form a primary yolk sac-like organoid. The epiblast and hypoblast are arranged in an orderly manner between the amniotic cavity and the yolk sac, forming an embryoid similar to embryos at CS5b and CS5c. In addition, in the testicular lumen on day 10, it can be observed that some embryoids start to develop toward gastruloids. Epi-like cells undergo epithelial-mesenchymal transition (EMT) to obtain EOMES/T positive and SOX2 negative gastrulation cells with reduced OCT4 expression.

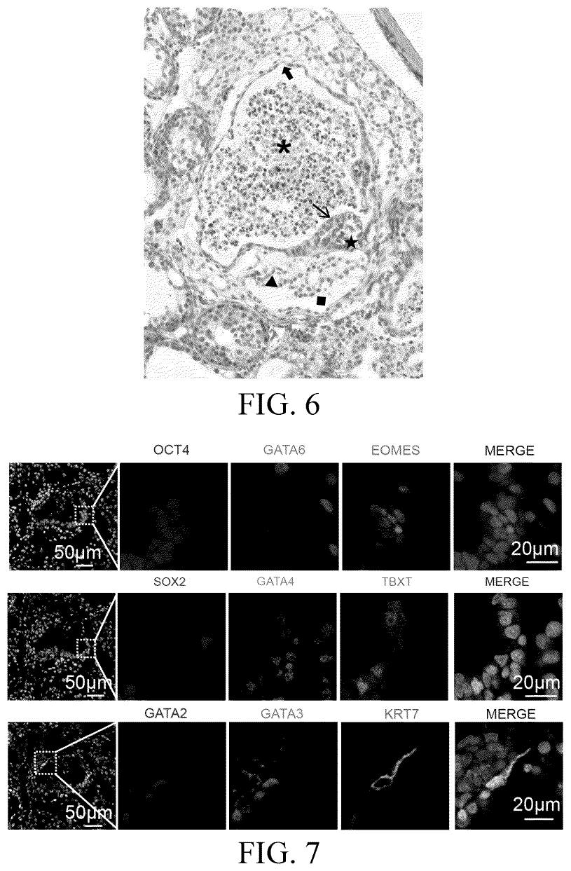

On day 20, gastrulation cells appear and form a gastruloid, to obtain a gastruloid model. OCT4 positive cells surround a cavity to further form an amniotic organoid, and gradually differentiate on the apical side of the amniotic organoid into KRT7/GATA2/GATA3 positive amniotic-like epithelial cells. In addition, EOMES/T positive gastrulation cells appear, and yolk sacs of some embryos are gradually covered by proliferating and migrating mesoendodermal cells (FIG. 6 and FIG. 7).

### Example 4: Formation of an Organoid Model by Injecting the Cell Line into a Mouse

Organoid development can be mimicked on day 30 to day 90 after the gastruloid stem cell line CCRM-hGOSC-1 in the present invention is injected into the testis of the mouse.

1. Injection into the Testis of the Nude Mouse

Preparation: A suitable capillary glass needle was made using a needle puller with parameters: Heat 515, Pull 100, Trip 75, delay 75.

When the confluency of the CCRM-hGOSC-1 cells reached 70%-90%, the medium was removed, the cells were washed with PBS (0.01 M, pH 7.4) at least twice to remove the spent medium and detached cells in poor condition, digested with 1-2 mL of EDTA-Trypsin, and observed under a microscope. The digested cells were collected during digestion. The digestion was stopped for the collected cells. Finally, all the cells were digested. The cells were centrifuged at 1000 rpm for 5 min. A supernatant was removed.

The cells were resuspended with a GK10 medium in a density of 2×10⁶ cells/mL. 5-6×10⁵ cells were injected into one testis of the BALB/c Nude mouse each time.

The mouse after injection was fed normally.

### 2. Validation of Organoid Formation

### 2.1 Testicular Tissue Sampling and Preparation

The testicular tissue of the mouse after injection in 1 was sampled every 10 days from day 30 to day 90 after the injection.

The sampled testicular tissue was placed in 4% PFA or mDF fixation solution for fixation at room temperature for 6 h, and then cut in half.

The fixation solution was removed after 42 hours. The testicular tissue was dehydrated at room temperature. Specifically, the testicular tissue was dehydrated with 70% alcohol for 24 h, dehydrated with 80% alcohol for 2 h, dehydrated with 90% alcohol for 2 h, dehydrated with 100% alcohol for 1 h, dehydrated with alcohol and xylene in 1:1 for 25 min, and cleared with xylene for 25 min.

The tissue block was transferred into an embedding cassette and infiltrated into paraffin (1) and paraffin (2) each for 45 min.

The embedded tissue was cut into consecutive 5 μm-thick sections. The paraffin sections were floated on a water bath to stretch the paraffin sections. Then, an intact tissue section was selected and lifted to adhere to a slide.

The slide was dried at 65° C. overnight. The slide was transferred to a 37° C. oven for 30 min and then stored at room temperature for a long time.

### 2.2 HE (Hematoxylin and Eosin) Staining

The paraffin section was deparaffinized and incubated in xylene (1) and xylene (2) each at 37° C. for 15 min.

The tissue was rehydrated in alcohol at room temperature in a gradient: 100% alcohol (1), 100% alcohol (2), 90% alcohol, 80% alcohol, and 70% alcohol, each for 2 min, and finally immersed in tap water for 10 min.

The section was stained in hematoxylin for 40 s and then rinsed under running water for 5 min.

The section was washed once with 1% HCl, and rinsed under running water for 10 min.

The section was stained in eosin for 3 min.

After staining, the tissue section was dehydrated in alcohol in a gradient: 70% alcohol, 80% alcohol, 90% alcohol, 100% alcohol (1), and 100% alcohol (2), each for 2 min, and in xylene (1) and xylene (2) each for 15 min.

The tissue on which resin was dropped was covered by a coverslip for cover slipping, placed in an oven at 37° C. for 2 h, then taken out and stored at room temperature, and then placed under an upright microscope for imaging.

### 2.3 Immunofluorescence (IF) Staining

The tissue section was deparaffinized, rehydrated, and transferred in tap water, which were the same as in HE staining.

Antigen retrieval: 200 mL of acidic antigen retrieval solution was prepared and added into an antigen retrieval box. The section was transferred into the retrieval box and then placed in a microwave at high power for 3 min and at low power for 7 min for antigen retrieval, and then naturally cooled down to room temperature.

Blocking: The slide was washed with PBS. A circle was drawn around the tissue using an immunohistochemical pen, and 100 μL of 5% BSA solution was added within the circle for incubation at room temperature for 2 h.

Incubation with primary antibody: The liquid on the tissue was aspirated as much as possible. The tissue was then covered by the antibody diluted proportionally with 5% BSA and incubated at 4° C. overnight.

The tissue with the antibody removed was washed with PBS three times, for 5 min each time.

Fluorescent secondary antibody and live cell staining solution Hoechst 33342 were diluted with 5% BSA at 1:1000, and added to the tissue slide for incubation at room temperature for 2 h. The slide was washed with PBS three times, for 5 min each time.

The slide was cover-slipped with glycerol. A confocal fluorescence microscope was used for imaging.

2.4 HE and IF Results of Organoids from Three Germ Layers

After the gastruloid stem cell line CCRM-hGOSC-1 in the present invention is injected into the testis of the mouse, on day 30 to day 40, the amniotic cavity further proliferates and enlarges, and neural ectoderm starts to appear, so that a neural ectoderm model is obtained; on day 90, CCRM-hGOSC-1 differentiates and forms a tissue or organ from ectoderm, mesoderm, and endoderm, such as neuroepithelium, smooth muscle, or intestines.

Endoderm: Through immunofluorescence anti-CDX2&GATA6 antibody labeling and HE staining morphology analysis, it can be learned that injected CCRM-hGOSC-1 gradually forms an intestinal structure with time, and forms a muscle-coated intestinal organoid on day 90, so that an intestinal organoid model is obtained.

Mesoderm: Through immunofluorescence SOX9 labeling cartilage and ACTA2 labeling muscle, with HE staining morphology analysis, it can be learned that CCRM-hGOSC-1 forms muscle and cartilage on day 90 of injection, so that a muscular organoid model and a cartilaginous organoid model are obtained.

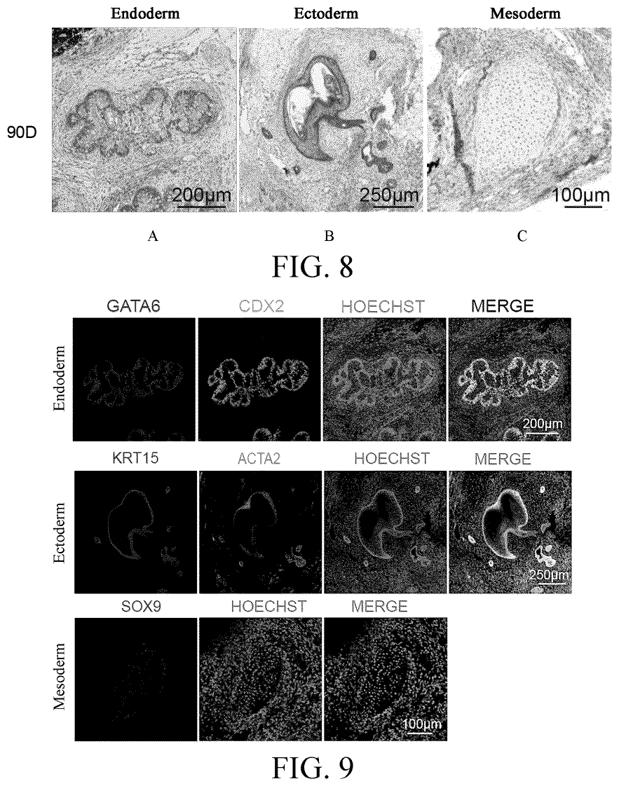

Ectoderm: 30 days after injection of CCRM-hGOSC-1 into the testicular lumen, a small number of primordial germ cells are found through SOX17, BLIMP1, and TFAP2C antibody labeling, so that a primordial germ cell model is obtained; and amniotic epithelial cells are found in the vicinity of the primordial germ cells through GATA2, GATA3, and KRT7 antibody labeling, so that an amniotic cavity model is obtained. With HE staining morphology, as well as KER15 indicating keratinocytes and ACTA2 indicating muscle, the skin structure is found on day 70 to day 90 of culture, so that a skin organoid model is obtained. Through OTX2 and SOX2 labeling neuroepithelial or radial glial cells, and TUJ1 and DCX labeling neurons, it can be learned through immunofluorescence staining that neuroepithelial cells with stemness appear on day 40 to day 50, so that a neuroepithelial cell model is obtained, and the neuroepithelial cells differentiate and form neurons on day 70 to day 90, so that a neural organoid model is obtained (FIG. 8 and FIG. 9).

In other words, a neural ectoderm model, a primordial germ cell model, and/or an amniotic epithelial cell model are/is obtained on day 30 to day 40 of the culture;
a neuroepithelial cell model is obtained on day 40 to day 50 of the culture; and
an intestinal organoid model, a muscular organoid model, a cartilaginous organoid model, a neural organoid model, and/or a skin organoid model are/is obtained on day 70 to day 90 of the culture.

In conclusion, the gastruloid stem cells CCRM-hGOSC-1 that can be passaged stably are established in this research. The cells maintain the pluripotency of stem cells to a certain extent, exhibit gene and protein expression of cells from three germ layers, endoderm, mesoderm, and ectoderm, during gastrulation, and have characteristics consistent with those of cells during gastrulation, so that key characteristics of cells during gastrulation can be well reproduced. The stem cell line can form a gastruloid model in the mouse, and can form an organoid model for a tissue or organ from ectoderm, mesoderm, and endoderm, such as neuroepithelium, smooth muscle, or intestines. Through this model, an in vitro platform for screening drugs affecting early embryonic development can be established, providing a reference for clinical medication.

### 2. Method for Constructing a Gastruloid Stem Cell Line by Inducing Human Induced Pluripotent Stem Cells

The present invention further discloses a method for inducing human induced pluripotent stem cells into the gastruloid stem cell line, and constructs a human in vitro post-implantation gastruloid model derived from the cell line. The reagents, instruments, and cell lines used in the present invention are commercially available. The human induced pluripotent stem cells are purchased from the Cell Bank/Stem Cell Bank of China Center for Type Culture Collection of Chinese Academy of Sciences, with the cell name of DYR0100 and catalog number of SCSP-1301.

(1) Sources of Components in the Medium Used for Inducing the Human Pluripotent Stem Cells into the Human Gastruloid Stem Cell Line In Vitro:
GMEM (Glasgow's MEM) medium: 11710035, purchased from Gibco, USA;
serum replacement (Thermo Fisher confidential formula): 10828028, purchased from Gibco, USA;
fetal bovine serum: 12483020, purchased from Gibco, USA;
Gibco® MEM non-essential amino acids: 11140076, purchased from Gibco, USA;
GlutaMAX™ additive: 35050061, purchased from Gibco, USA;
sodium pyruvate additive: 11360070, purchased from Gibco, USA;
penicillin-streptomycin double antibiotic solution: 15140122, Gibco, USA;
β-mercaptoethanol: 21985023, purchased from Gibco, USA;
recombinant human bone morphogenetic protein 4:314-BP, purchased from R&D Systems, USA;
recombinant human stem cell factor: 7734-LF, purchased from R&D Systems, USA;
recombinant human leukemia inhibitory factor: 225-SC, purchased from R&D Systems, USA;
recombinant human epidermal growth factor: 236-EG, purchased from R&D Systems, USA;
recombinant human activin A protein: 338-AC, purchased from R&D Systems, USA;
ROCK inhibitor: specifically Y-27632, HY-10071, purchased from MCE, USA;
CHIR 99021: specifically Laduviglusib trihydrochloride, HY-10182B, purchased from MCE, USA;
adenylate cyclase activator: specifically forskolin, 1099, purchased from R&D Systems, USA; and
PDE4 inhibitor: specifically Rolipram, 0905, purchased from R&D Systems, USA.
   (2) Sources of Components in the Medium Used for Inducing the Human Pluripotent Stem Cells into the Post-Implantation Blastoid In Vitro:
mTeSR™1 medium: #85850, purchased from STEMCELL Technologies, Canada;
Essential 6 medium: A1516401, purchased from Gibco, USA;
recombinant human fibroblast growth factor 2:3718-FB, purchased from R&D Systems, USA;
recombinant human Noggin protein: HY-P7051A, purchased from MCE, USA;
ROCK inhibitor: specifically Y-27632, HY-10071, purchased from MCE, USA; and
WNT inhibitor: specifically IWP-2, S7085, purchased from Selleck, USA.
   (3) Preparation of a GK15-1 Medium Containing Y-27632 at Stage 1 of Induction from Pluripotent Stem Cells to Mesoderm:

81% of basal medium GMEM in percentage by volume, 15% of serum replacement KOSR in percentage by volume, 1% of penicillin-streptomycin double antibiotic solution (containing 10000 units/mL penicillin and 10000 μg/mL streptomycin) in percentage by volume, 1% of 10 mM non-essential amino acids in percentage by volume, 1% of 200 mM GlutaMAX additive in percentage by volume, 1% of 100 mM sodium pyruvate additive in percentage by volume, 0.1 mM β-mercaptoethanol, 50 ng/mL recombinant human activin A, 3 μM glycogen synthase kinase-3 (GSK-3) inhibitor CHIR 99021, and 10 μM ROCK inhibitor Y-27632.

(4) Preparation of a GK15-1 Medium in Induction from Pluripotent Stem Cells to Mesoderm (Stage 1):

81% of basal medium GMEM in percentage by volume, 15% of serum replacement KOSR in percentage by volume, 1% of penicillin-streptomycin double antibiotic solution (containing 10000 units/mL penicillin and 10000 μg/mL streptomycin) in percentage by volume, 1% of 10 mM non-essential amino acids in percentage by volume, 1% of 200 mM GlutaMAX additive in percentage by volume, 1% of 100 mM sodium pyruvate additive in percentage by volume, 0.1 mM β-mercaptoethanol, 50 ng/mL recombinant human activin A, and 3 μM glycogen synthase kinase-3 (GSK-3) inhibitor CHIR 99021.

(5) Preparation of a GK15-2 Medium Containing Y-27632 in Induction from Nascent Mesoderm-Like Cells to Primordial Germ Cell-Like Cells (Stage 2) and in Cell Purification:

81% of basal medium GMEM in percentage by volume, 15% of serum replacement KOSR in percentage by volume, 1% of penicillin-streptomycin double antibiotic solution (containing 10000 units/mL penicillin and 10000 μg/mL streptomycin) in percentage by volume, 1% of 10 mM non-essential amino acids in percentage by volume, 1% of 200 mM GlutaMAX additive in percentage by volume, 1% of 100 mM sodium pyruvate additive in percentage by volume, 0.1 mM β-mercaptoethanol, 100 ng/mL recombinant human stem cell factor, 200 ng/mL recombinant human bone morphogenetic protein 4, 1000 U/mL recombinant human leukemia inhibitory factor, 50 ng/mL recombinant human epidermal growth factor, and 10 μM ROCK inhibitor Y-27632.

(6) Preparation of a GK15-2 Medium in Induction from Nascent Mesoderm-Like Cells to Primordial Germ Cell-Like Cells (Stage 2) and in Cell Purification:

81% of basal medium GMEM in percentage by volume, 15% of serum replacement KOSR in percentage by volume, 1% of penicillin-streptomycin double antibiotic solution (containing 10000 units/mL penicillin and 10000 μg/mL streptomycin) in percentage by volume, 1% of 10 mM non-essential amino acids in percentage by volume, 1% of 200 mM GlutaMAX additive in percentage by volume, 1% of 100 mM sodium pyruvate additive in percentage by volume, 0.1 mM β-mercaptoethanol, 200 ng/ml recombinant human bone morphogenetic protein 4, 100 ng/mL recombinant human stem cell factor, 1000 U/mL recombinant human leukemia inhibitory factor, and 50 ng/mL recombinant human epidermal growth factor.

### (7) Preparation of a GK10 Medium Containing Y-27632:

83.5% of basal medium GMEM in percentage by volume, 10% of serum replacement KOSR in percentage by volume, 2.5% of fetal bovine serum FBS in percentage by volume, 1% of penicillin-streptomycin double antibiotic solution (containing 10000 units/mL penicillin and 10000 μg/mL streptomycin) in percentage by volume, 1% of 10 mM non-essential amino acids in percentage by volume, 1% of 200 mM GlutaMAX additive in percentage by volume, 1% of 100 mM sodium pyruvate additive in percentage by volume, 0.1 mM β-mercaptoethanol, 10 UM forskolin, 10 μM Rolipram, 100 ng/mL recombinant human stem cell factor, and 10 μM ROCK inhibitor Y-27632.

### (8) Preparation of a GK10 Medium in the In Vitro Expansion of a Gastruloid Stem Cell Line:

83.5% of basal medium GMEM in percentage by volume, 10% of serum replacement KOSR in percentage by volume, 2.5% of fetal bovine serum FBS in percentage by volume, 1% of penicillin-streptomycin double antibiotic solution (containing 10000 units/mL penicillin and 10000 μg/mL streptomycin) in percentage by volume, 1% of 10 mM non-essential amino acids in percentage by volume, 1% of 200 mM GlutaMAX additive in percentage by volume, 1% of 100 mM sodium pyruvate additive in percentage by volume, 0.1 mM β-mercaptoethanol, 10 μM forskolin, 10 μM Rolipram, and 100 ng/mL recombinant human stem cell factor.

### (9) Preparation of an mTR Medium at Stage 1 of Post-Implantation Gastruloid Induction:

99% of mTeSR™ 1 complete medium in percentage by volume, 1% of double antibiotic solution containing 10000 units/mL penicillin and 10000 μg/mL streptomycin in percentage by volume, and a small molecule compound: 10 μM ROCK inhibitor Y-27632.

### (10) Preparation of an E6BIN Medium at Stage 2 of Post-Implantation Gastruloid Induction:

100% of Essential 6 medium in percentage by volume, and multiple cytokines: 20 ng/ml recombinant human fibroblast growth factor 2, 50 ng/mL recombinant human Noggin protein, and 5 μM small molecule compound IWP-2.

### Example 5: Construction of the Gastruloid Stem Cell Line DYR0100-hGOSC-1

This example of the present invention provides gastruloid stem cells named human gastruloid stem cell line DYR0100-hGOSC-1. The cell line is deposited in the China Center for Type Culture Collection with an accession number of CCTCC NO. C2022115.

(1) Induction from Human Induced Pluripotent Stem Cells to Mesoderm (Stage 1):

(1-1) The human induced pluripotent stem cells DYR0100 are purchased from the Cell Bank/Stem Cell Bank of China Center for Type Culture Collection of Chinese Academy of Sciences, with the catalog number of SCSP-1301.

The human induced pluripotent stem cells were digested into single cells with TrypLE Select when the cells grew to 80%-90% confluency, and centrifuged by using a horizontal centrifuge at 1300 rpm for 3 min. Then, the cells were resuspended with PBS to obtain a cell suspension 1.

(1-2) The cell suspension 1 was centrifuged, a supernatant was removed, and a GK15-1 medium containing Y-27632 was added to further resuspend cells, to obtain a cell suspension 2. Each 1 mL of the GK15-1 medium containing Y-27632 contained 1×10⁶ cells.

(1-3) The cell suspension 2 obtained in (1-2) was inoculated into a six-well plate pre-coated with matrigel in a density of 1×10⁶ cells/cm², shaken uniformly, and then placed in an incubator at 37° C. under carbon dioxide in a volume concentration of 5.0%.

(1-4) On day 2 of the culture, the spent medium was replaced with a GK15-1 medium. The medium was changed daily until nascent mesoderm-like cells were obtained.

The foregoing steps of induction from the pluripotent stem cells to mesoderm were implemented on day 0 to day 2 of the construction of the gastruloid stem cell line.

(2) Induction from the Nascent Mesoderm-Like Cells to Primordial Germ Cell-Like Cells (Stage 2):

(2-1) The nascent mesoderm-like cells obtained in (1-4) were digested into single cells with TrypLE Select when the cells grew to 80%-90% confluency, and centrifuged by using a horizontal centrifuge at 1300 rpm for 3 min. Then, the cells were resuspended with PBS to obtain a cell suspension 3.

(2-2) The cell suspension 3 was centrifuged, a supernatant was removed, and a GK15-2 medium containing Y-27632 was added to further resuspend cells, to obtain a cell suspension 4. Each 1 mL of the GK15-2 medium containing Y-27632 contained 1×10⁵ cells.

(2-3) The cell suspension 4 obtained in (2-2) was inoculated into a transparent low-attachment 96-well plate with round bottoms for sphere-forming culture, where cell spheres in each well initially contained 1×10⁴ cells. On day 2 of the culture, the spent medium was replaced with a GK15-2 medium. The medium was replaced with a GK15-2 medium daily until cell spheres containing primordial germ cell-like cells were obtained.

The foregoing steps of induction from the nascent mesoderm-like cells to the primordial germ cell-like cells were implemented on day 3 to day 6 of the construction of the gastruloid stem cell line.

### (3) Cell Purification:

(3-1) The cell spheres obtained in (2-3) were digested into single cells with collagenase IV and 0.25% Trypsin-EDTA trypsin, and centrifuged by using a horizontal centrifuge at 1300 rpm for 3 min. The cells were resuspended with a GK15-2 medium to obtain a cell suspension 5.

(3-2) CD326 and CD49f double-positive cells were separated by using a flow cytometer from the cell suspension 5 obtained in (3-1), centrifuged at 1300 rpm for 3 min, and inoculated into a 12-well plate in a density of 1.0×10⁵ cells per well. 1 mL of GK10 medium containing Y-27632 was added to cells in each well to resuspend the cells, to obtain a cell suspension 6.

### (4) Cell Expansion:

The cell suspension 6 obtained in (3-2) was inoculated in a density of 2×10⁴ cells/cm² into a 12-well plate in which mouse embryo fibroblasts treated with mitomycin C were pre-spread as feeder cells, shaken uniformly, and placed in an incubator at 37° C. under carbon dioxide in a volume concentration of 5.0%. The medium was replaced with a fresh GK10 medium after 24 hours. The medium was changed daily. The gastruloid stem cell line DYR0100-hGOSC-1 was obtained when formed clones can be observed, which was deposited in the China Center for Type Culture Collection with an accession number of CCTCC NO. C2022115.

### Example 6: Description of Biological Characteristics of Cells

### 1.1 Cell Morphology Observation

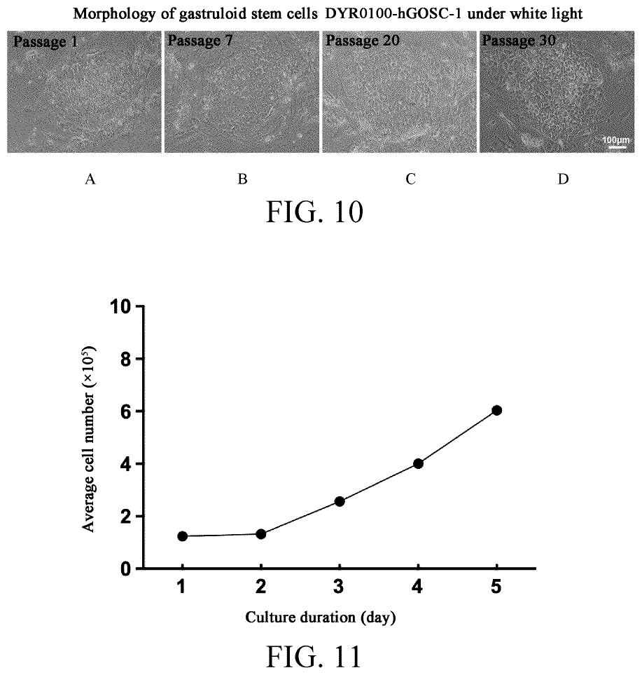

The gastruloid stem cells DYR0100-hGOSC-1 were observed under an upright microscope, which were round or oval. The cells proliferate rapidly, clones are formed visibly on day 2 or day 3 after single-cell passage, and the cell clones have clear boundaries. The morphology observation results are shown in FIG. 10.

### 1.2 Growth Curve Determination

### 1.2.1 Steps of Growth Curve Determination

The operations are the same as those in 1.2.1 Steps of growth curve determination in Example 2, except that the cells are replaced with the gastruloid stem cells DYR0100-hGOSC-1.

### 1.2.2 Results of Growth Curve Determination

The determination was carried out for five consecutive days to obtain growth curve data shown in Table 2 below.

Table 2 shows the growth curve data obtained through five consecutive days of determination.

TABLE 2

| | |
|---|---|
| Culture duration (day) | Average cell number (×10⁵) |

| | | |
|---|---|---|
| 1 | day | 1.24 × 10⁵ |
| 2 | days | 1.32 × 10⁵ |
| 3 | days | 2.56 × 10⁵ |
| 4 | days | 4.00 × 10⁵ |
| 5 | days | 6.04 × 10⁵ |

A cell growth curve shown in FIG. 11 is obtained based on the cell growth curve data in Table 2. In FIG. 11, horizontal coordinates indicate the culture duration (day), and vertical coordinates indicate the cell number (×10⁵).

With reference to the growth curve data in Table 2 and the growth curve shown in FIG. 11, it can be learned that the gastruloid stem cells DYR0100-hGOSC-1 grew well for the five consecutive days, and a logarithmic growth phase was three days.

In conclusion, the DYR0100-hGOSC-1 cells proliferate rapidly, and exhibit active growth, good activity, high culture stability, and stable cell growth characteristics for in vitro culture.

### 1.3 Immunofluorescence Identification

### 1.3.1 Steps of Immunofluorescence Identification

A small round coverslip was sterilized with 75% ethanol and by ultraviolet light, and then transferred into a cell culture dish. The coverslip was coated with Fibronectin to improve attachment. Then, the DYR0100-hGOSC-1 cells were inoculated and cultured according to steps of normal cell passage culture.

Before passage of the cultured DYR0100-hGOSC-1 cells, the small round coverslip was taken out and placed in a new clean culture dish. The DYR0100-hGOSC-1 cells on the small round coverslip were fixed with 4% PFA at room temperature for 30 min.

The 4% PFA fixation solution was removed, and then the remaining fixation solution was washed off with PBS at room temperature three times, for 5 min each time. The PBS was removed. The 5% BSA blocking buffer was added for blocking at room temperature for 2 h.

The 5% BSA blocking buffer was removed. With the corresponding primary antibody diluted proportionally in 5% BSA added, the DYR0100-hGOSC-1 cells on the small round coverslip were incubated at 4° C. overnight.

After the primary antibody was removed, the remaining primary antibody was washed off with PBS at room temperature three times, for 5 min each time. The cells were incubated in the dark at room temperature for 2 h with the secondary antibody and live cell staining solution Hoechst 33342 that were diluted with 5% BSA and added to the dish.

After the secondary antibody was removed, the remaining secondary antibody was washed off with PBS three times, for 5 min each time. Glycerol was added onto a slide. The small round coverslip was taken out of the culture dish and inverted onto the slide with glycerol added, and then fixed with nail polish. A confocal fluorescence microscope was used for imaging.

### 1.3.2 Results of Immunofluorescence Identification

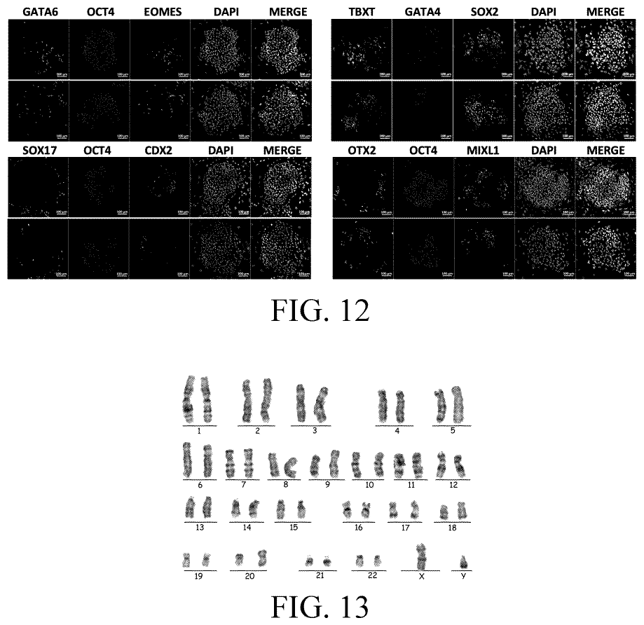

The results of immunofluorescence identification are shown in FIG. 12. The gastruloid stem cells DYR0100-hGOSC-1 were subjected to immunofluorescence staining through cell growing on a coverslip, to identify the expression of genes in each germ layer in the DYR0100-hGOSC-1 cells. It is found that pluripotency genes OCT4 and SOX2, mesoderm genes EOMES, TBXT, MIXL1, and CDX2, and endoderm genes GATA4, GATA6, SOX17, FOXA2, and OTX2 are expressed in cell clones. In conclusion, the pluripotency genes are expressed in the center of the cell clones, the mesoderm genes are expressed dispersedly inside the cell clones, and the endoderm genes are expressed at the edges of the cell clones and are not colocalized with the pluripotency genes.

### 1.4 Karyotyping Identification

### 1.4.1. Steps of Karyotyping Identification

20 μg/mL colchicine was added to the GK10 medium of the gastruloid stem cells at 1:200 to reach a final concentration of 0.1 μg/mL. Then, the cells were placed in an incubator at 37° C. under carbon dioxide in a volume concentration of 5.0% for incubation for 3 h.

A 0.56% KCl hypotonic solution was pre-warmed to 37° C. The culture dish was taken out. The colchicine-treated gastruloid stem cells were washed with PBS (0.01 M, pH 7.4) at least three times to remove the spent medium and detached cells in poor condition. Then, the gastruloid stem cells DYR0100-hGOSC-1 were digested with TrypLE Select into a single-cell suspension. The cell suspension was transferred to a 15 mL centrifuge tube and centrifuged by using a horizontal centrifuge at 2000 rpm for 10 min. After a supernatant was removed, to the cell pellet was added 9 mL of the 0.56% KCl hypotonic solution pre-warmed to 37° C. The cell pellet was pipetted gently using a glass dropper with a rubber bulb for about 50 times until a single-cell suspension was obtained, to carry out hypotonic treatment in a water bath at 37° C. for 25 min.

Methanol and glacial acetic acid were prepared at 3:1 and mixed uniformly at room temperature to obtain a fixation solution. The gastruloid stem cell suspension after the hypotonic treatment, with 1 mL of the fixation solution added, was pipetted gently from top to bottom using a glass dropper with a rubber bulb for 20 times, and then centrifuged at 2000 rpm for 10 min. A supernatant was removed. With 10 mL of fresh fixation solution added, the cells were pipetted gently from top to bottom using a glass dropper with a rubber bulb for 10 times into a single-cell suspension, to carry out fixation at room temperature for 2 h, and then centrifuged at 2000 rpm for 10 min. A supernatant was removed. With 10 mL of fresh fixation solution added, the cells were pipetted gently from top to bottom using a glass dropper with a rubber bulb for 10 times into a single-cell suspension, to carry out fixation at room temperature for 30 min, and then centrifuged at 2000 rpm for 10 min. A supernatant was removed. 0.2-0.6 mL of fixation solution was added based on the amount of cells precipitated to resuspend the gastruloid stem cells.

An alcohol lamp was lighted. A clean slide was taken out of distilled water without drying, and placed obliquely on a waste tank, while the cell suspension 50 cm above the slide was dropped on the slide using a pipette. One drop of the cell suspension was placed at each of three or four different positions of each slide, followed immediately by cauterizing the slide on the back by the alcohol lamp five times. The slide was transferred to an oven at 37° C. for overnight baking.

The slide with the human gastruloid stem cell chromosome sample was taken out of the oven at 37° C. and placed in an oven at 80° C. for 2.5 h. 0.25% Trypsin-EDTA was pre-warmed to 37° C. for use. The slide was immersed in 0.25% Trypsin-EDTA at 37° C. for 30-40 s, and then taken out and rinsed on both sides under running water three times. The slide was immersed in Giemsa stain pre-warmed to 37° C. for staining for about 10 min, then rinsed on both sides under running water three times, and wiped using lens wiping paper until the surface was dried.

Well-dispersed division phases of moderate lengths were selected under a low-power microscope, and then observed using a lens immersed in oil and imaged, to obtain results of karyotyping identification.

### 1.4.2 Results of Karyotyping Identification

The results of karyotyping identification are shown in FIG. 13. With reference to FIG. 13, through the karyotyping identification, it can be learned that the cells exhibit normal chromosome structures and quantities, and the cells have 44+XY chromosomes and belong to a diploid karyotype male cell line.

### 1.5 RNA Sequencing Identification

### 1.5.1 Steps of RNA Sequencing Identification

The operations are the same as those in 1.5.1 in Example 1, except that the cells are replaced with the gastruloid stem cells DYR0100-hGOSC-1.

### 1.5.2 Results of RNA Sequencing Identification

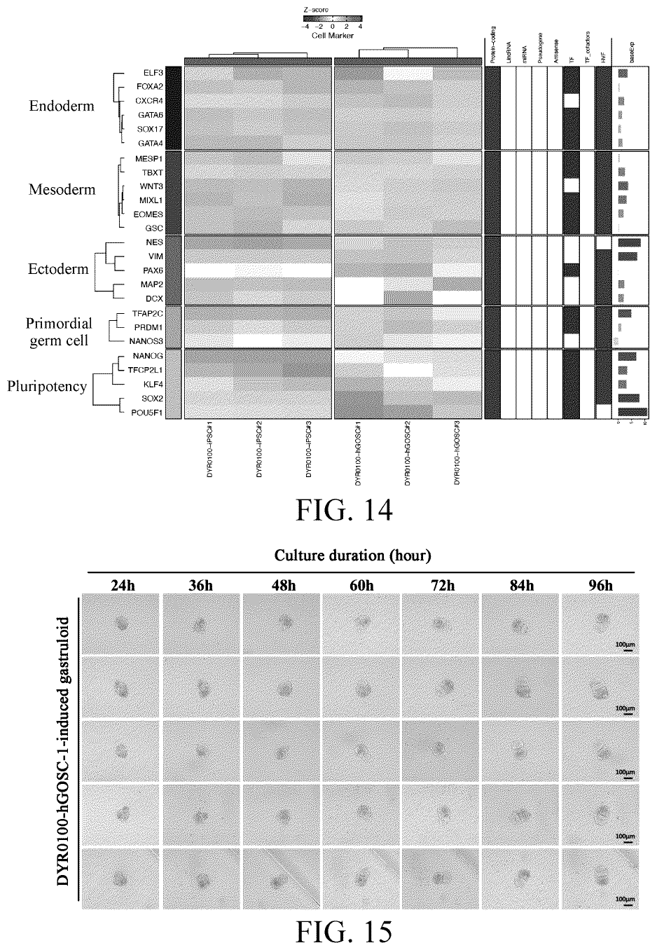

The results of RNA sequencing identification are shown in FIG. 14. Through the comparison in transcriptome sequencing between the gastruloid stem cells DYR0100-hGOSC-1 and the human induced pluripotent stem cells DYR0100-hiPSCs, it can be found that, compared with the human induced pluripotent stem cells DYR0100-hiPSCs, the gastruloid stem cells DYR0100-hGOSC-1 exhibit upregulation of the expression of both the mesoderm genes MIXL1, EOMES, MESP1, WNT3, TBXT, and GSC and the endoderm genes ELF3, FOXA2, CXCR4, GATA4, GATA6, and SOX17.

In the gastruloid stem cells DYR0100-hGOSC-1, compared with the human induced pluripotent stem cells DYR0100-hiPSCs, the expression of the pluripotency gene SOX2 is slightly downregulated, and the other pluripotency genes POU5F1 (OCT4) and NANOG are still expressed. In addition, the expression of naïve pluripotency factors KLF4 and TFCP2L1 upregulated in the human induced pluripotent stem cells increases in the gastruloid stem cells DYR0100-hGOSC-1, indicating that the gastruloid stem cell line DYR0100-hGOSC-1 still exhibits pluripotency of stem cells.

The gastruloid stem cells DYR0100-hGOSC-1 maintain the pluripotency of stem cells to a certain extent, and exhibit gene expression of lineage cells from multiple germ layers, with characteristics similar to those of embryonic cells during gastrulation in human embryonic development.

### Example 7: Induction from the Gastruloid Stem Cells DYR0100-hGOSC-1 to a Post-Implantation Three-Dimensional Gastruloid Model (Stage 1, Day 0 and Day 1)

The gastruloid stem cells DYR0100-hGOSC-1 have characteristics of a human embryo during gastrulation, which can mimic gastrulation for studying morphologic developmental characteristics and genetic functions during gastrulation. Through in vitro culture of the cell line, a structure similar to the in vivo gastrula can be reconstituted, and biological events during gastrulation can be partially reproduced.

When the gastruloid stem cells obtained in Example 5 grew to 70%-80% confluency, the cells were digested with TrypLE Select into single cells, and the cells were resuspended with GK10, to obtain a cell suspension 7. CD326 and CD49f double-positive cells were separated by using a flow cytometer, and centrifuged at 1300 rpm for 3 min. 6.0-7.0×10⁴ cells were taken and resuspended with 4 mL of mTR medium, re-inoculated into a transparent low-attachment 96-well plate with round bottoms with 6.0-7.0×10³ cells per well, centrifuged by using a horizontal centrifuge at 800 rpm for 3 min, and placed in an incubator at 37° C. under carbon dioxide in a volume concentration of 5.0%, until the gastruloid stem cells were assembled to form a three-dimensional structure.

### Example 8: Induction from the Gastruloid Stem Cells DYR0100-hGOSC-1 to a Post-Implantation Three-Dimensional Gastruloid Model (Stage 2, Day 2 to Day 4)

An E6BIN medium was added without removing the mTR medium in the well plate in Example 7, so that a final medium contained 20 ng/mL recombinant human fibroblast growth factor 2, 50 ng/mL recombinant human Noggin protein, and a small molecule compound: 5 μM IWP-2. The well plate was placed in an incubator at 37° C. under carbon dioxide in a volume concentration of 5.0%, until a proamniotic cavity was formed and mesoendodermal lineage specialization was completed, to obtain the gastruloid model.

### Example 9: Description of Biological Characteristics of the Gastruloid Model Obtained Through Induction from the Gastruloid Stem Cells

### 1.1 Morphology Observation of Gastruloid Induction

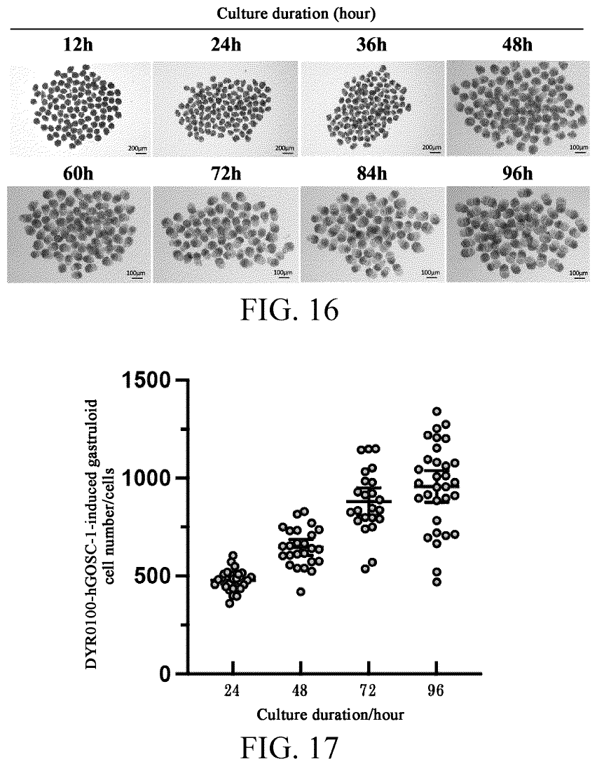

The post-implantation gastruloid model cultured in the 96-well plate for 0-96 hours was observed under an upright microscope. It can be seen that the cells form spheres rapidly, and form three-dimensional embryoid cell spheres 12 hours after the induction. Then, the cell spheres increase in volume slowly, and a region with dense cells and a region with loose cells appear inside the cell spheres after 48-60 hours, forming two mutually exclusive spheres. Then, the amniotic organoid is formed. A maximum diameter of the gastruloid after 96 hours is about 150-250 μm. The morphology observation results are shown in FIG. 15 and FIG. 16.

### 1.2 Growth Curve Determination of Post-Implantation Gastruloid Induction

### 1.2.1 Steps of Growth Curve Determination

During day 1 to day 4 of in vitro construction of the post-implantation gastruloid model from the gastruloid stem cells DYR0100-hGOSC-1, sampling was carried out every 24 hours. The sampled gastruloid was washed with PBS (0.01 M, pH 7.4) at least twice to remove the spent medium and detached cell debris in poor condition, and then fixed with 4% PFA at room temperature for 30 min. Then, the fixation solution was removed, and the gastruloid was washed with PBST (0.01 M, pH 7.4, containing 1% tTriton) at least three times, for 5 min each time.

The gastruloid was blocked with 5% BSA (containing 1% tTriton) at room temperature for 4 h, and then incubated at room temperature for 4 h in DAPI diluted in 5% BSA at 1:200.

After the diluted DAPI was removed, the gastruloid was washed with PBS three times, for 5 min each time. The sample was transferred to a staining chamber, and cleared with a specific amount of prepared iohexol solution. The chamber was covered with a slide and then placed under a confocal microscope for imaging.

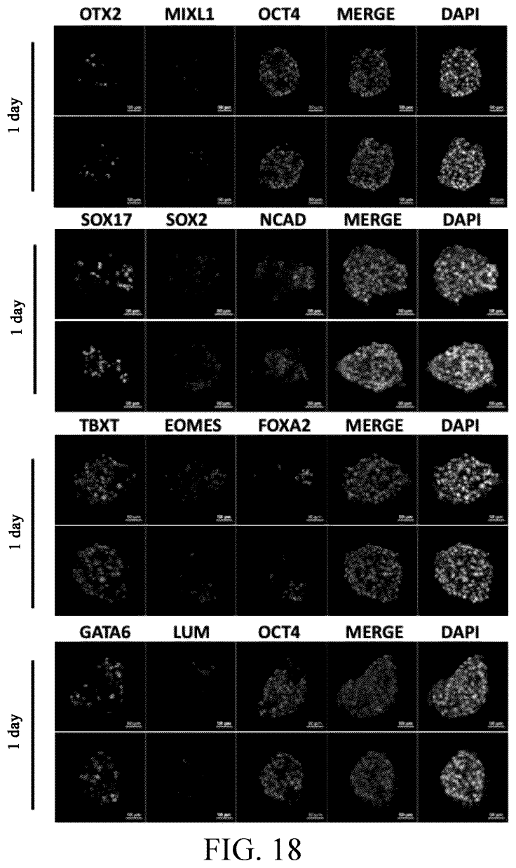

A cell number inside each cell sphere was estimated on the DAPI-stained confocal image by using the Imaris software (Bitplane). Count points were plotted using the internal algorithm, with an estimated xy size of 6-10 μm, a quality threshold of 2.5, and background subtraction (FIG. 17).

### 1.2.2 Results of Growth Curve Determination

The determination was carried out for four consecutive days to obtain cell counting data inside the cell sphere at each time point shown in Table 3 below.

TABLE 3

| | Culture duration (hour) | | | |
|---|---|---|---|---|
| | 24 hours | 48 hours | 72 hours | 96 hours |
| | (n = 28) | (n = 25) | (n = 23) | (n = 31) |
| Sample cell number | 428 | 708 | 1149 | 1044 |
| (cells) | 516 | 555 | 799 | 1008 |
| | 604 | 737 | 751 | 1062 |
| | 494 | 576 | 1033 | 1274 |
| | 459 | 541 | 889 | 1219 |
| | 512 | 751 | 792 | 978 |
| | 493 | 733 | 826 | 885 |
| | 478 | 770 | 741 | 783 |
| | 506 | 612 | 914 | 1202 |
| | 490 | 666 | 837 | 1077 |
| | 459 | 603 | 571 | 916 |
| | 456 | 540 | 1144 | 1207 |
| | 435 | 829 | 1148 | 1153 |
| | 486 | 636 | 1052 | 1341 |
| | 487 | 731 | 798 | 695 |
| | 509 | 815 | 929 | 707 |
| | 399 | 652 | 834 | 720 |
| | 361 | 668 | 922 | 665 |
| | 436 | 606 | 984 | 713 |
| | 445 | 524 | 782 | 953 |
| | 397 | 573 | 847 | 1012 |
| | 572 | 615 | 978 | 1096 |
| | 509 | 641 | 537 | 469 |
| | 519 | 654 | — | 521 |
| | 457 | 419 | — | 974 |
| | 435 | — | — | 956 |
| | 482 | — | — | 1253 |
| | 549 | — | — | 1080 |
| | — | — | — | 910 |
| | — | — | — | 897 |
| | — | — | — | 897 |

Table 3 shows the growth curve data of the DYR0100-hGOSC-1-induced gastruloid obtained through four consecutive days of determination, and a quantity (n) of samples for counting at each time point is 20-30.

A cell growth curve shown in FIG. 17 is obtained based on the cell growth curve data in Table 3. In FIG. 17, horizontal coordinates indicate the culture duration (hour), and vertical coordinates indicate the cell number (cells).

With reference to the growth curve data in Table 3 and the growth curve shown in FIG. 17, it can be learned that, in the gastruloid stem cell DYR0100-hGOSC-1-induced gastruloid, the cells proliferate slowly, and exhibit high induction stability and stable embryoid growth characteristics for in vitro induction.

### 1.3 Immunofluorescence Identification

### 1.3.1 Steps of Immunofluorescence Identification

During day 1 to day 4 of induction from the gastruloid stem cells DYR0100-hGOSC-1 to the gastruloid model, sampling was carried out every 24 hours.

The sampled gastruloid was fixed with 4% PFA for 30 min, and then the fixation solution was removed. The gastruloid was washed with PBST (0.01 M, pH 7.4, containing 1% tTriton) three times, for 5 min each time.

The gastruloid was blocked with 5% BSA (containing 1% tTriton) at room temperature for 4 h.

The blocking buffer was removed. The gastruloid was incubated at 4° C. for 24-48 h with an antibody diluted proportionally.

After the primary antibody was removed, the gastruloid was washed with PBS (0.01 M, pH 7.4, containing 1% tTriton) three times, for 5 min each time. Then, the gastruloid was incubated with diluted secondary antibody and DAPI at room temperature for 4 h.

After the secondary antibody was removed, the gastruloid was washed with PBS (0.01 M, pH 7.4, containing 1% tTriton) three times, for 5 min each time. The sample was transferred to a silica gel staining chamber with a depth of 0.2 mm or 0.75 mm, and cleared with a specific amount of prepared iohexol solution. The chamber was covered with a slide and then placed under a confocal microscope for imaging, to obtain results of immunofluorescence staining.

### 1.3.2 Results of Immunofluorescence Identification

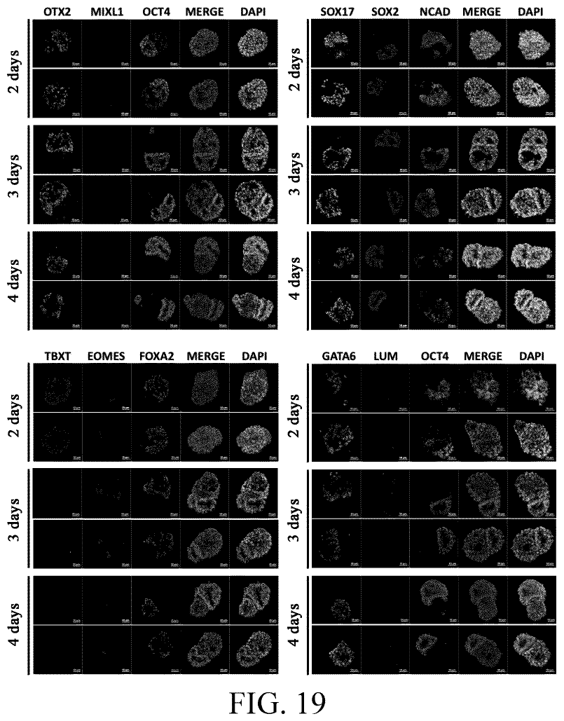

The results of immunofluorescence identification are shown in FIG. 18 and FIG. 19.

On day 1 of induction from the gastruloid stem cells to the gastruloid, the gastruloid stem cells start to assemble and form a stable three-dimensional cell sphere structure. In this case, inside the gastruloid, there are epiblast-like cells (expressing OCT4 and SOX2), primitive streak-like cells (expressing NCAD, TBXT, and MIXL1), mesoendoderm-like cells (expressing EOMES, GATA6, and NCAD), and endoderm-like cells (expressing NCAD, SOX17, OTX2, and FOXA2), and there are also a small number of extraembryonic mesoderm-like cells (expressing LUM). In this case, there is no clear pattern of distribution of the various cells in terms of spatial location.

On day 2 of induction, OCT4 and SOX2 positive epiblast-like cells start to migrate to one side of the gastruloid, SOX17, FOXA2, and OTX2 indicated endoderm-like cells also migrate in the opposite direction, MIXL1 and TBXT positive primitive streak-like cells also migrate in the direction opposite to the epiblast-like cells, but MIXL1 and TBXT positive primitive streak-like cells are reduced from day 1 of gastruloid induction, indicating that the primitive streak-like cells may start to specialize to mesoderm-like or definitive endoderm-like cells.

On day 3 and day 4 of induction, OCT4 and SOX2 positive epiblast-like cells form a cavity similar to the amniotic cavity. Compared with day 2 of gastruloid induction, SOX17, FOXA2, and OTX2 positive endoderm-like cells and OCT4 and SOX2 positive epiblast-like cells are completely separated and form a well-aligned bilaminar embryonic disc. The nuclei of the OCT4 and SOX2 positive epiblast-like cells are columnar and three-dimensional. The SOX17, FOXA2, and OTX2 positive endoderm-like cells and the EOMES positive FOXA2 negative mesoderm-like cells exhibit expression of the epithelial-mesenchymal transition marker NCAD signal.

### 1.5 RNA Sequencing Identification

### 1.5.1 Steps of RNA Sequencing Identification

At hour 96 of induction from the gastruloid stem cells DYR0100-hGOSC-1 to the gastruloid model, the gastruloid was sampled with the medium removed. The sampled gastruloid was washed with PBS (0.01 M, pH 7.4) at least twice to remove the spent medium and detached dead cell debris. The gastruloid was digested with 1-2 mL of 0.25% Trypsin-EDTA at 37° C. for 3 min. During the digestion, the gastruloid was pipetted gently by using a pipette into single cells, and then a serum medium was added to stop the digestion. The cells were centrifuged at 1300 rpm for 5 min, and then a supernatant was removed. The cells were resuspended with PBS and then transferred into an enzyme-free EP tube for sequencing.

### 1.5.2 Results of RNA Sequencing Identification

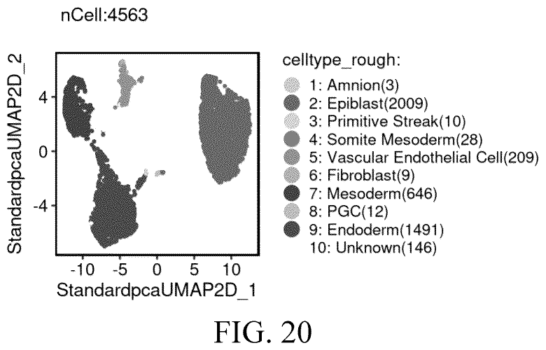

The results of single-cell RNA sequencing identification are shown in FIG. 20.

In the single-cell RNA sequencing data of the gastruloid model cultured for 96 hours, through uniform manifold approximation and projection (UMAP) analysis of 4563 cells, the cells are grouped into 12 cell clusters.

Based on the gene expression in each cell cluster, nine types of cells are finally identified: amnion cells, epiblast cells, somite mesoderm cells, vascular endothelial cells, fibroblasts, mesoderm cells, primordial germ cell-like cells (PGCs), endoderm cells, and some unknown cells. Consistent with the immunofluorescence images in FIG. 19, only 10 MIXL1 and TBXT expressing primitive streak cells were identified at these time points.

In this dataset, no cell types that express two or more neural ectoderm markers are detected.

These data indicate that in the embryoid model after 96 hours, epiblast cells have transiently undergone gastrulation, and the formed primitive streak cells have completed the differentiation into definitive endoderm and mesoderm, while neural differentiation has not begun. It indicates that the embryoid at this stage may be close to the embryo at Carnegie Stage 7 of embryonic development.

In conclusion, the gastruloid stem cells that can be passaged stably are established in this research. The cells maintain the pluripotency of stem cells to a certain extent, exhibit gene and protein expression of cells from three germ layers, endoderm, mesoderm, and ectoderm, during gastrulation, and have characteristics consistent with those of embryonic cells during gastrulation, so that key characteristics of cells during gastrulation can be well reproduced. Through the use of this type of cells, a three-dimensional gastruloid model that can mimic gastrulation can be constructed in vitro, and key biological events such as lineage separation of endoderm and ectoderm, formation of proamniotic cavity, emergence of primitive streak, and mesodermal lineage specialization during embryonic development in vivo can be partially reproduced. In combination with single-cell multi-omics sequencing and fluorescence imaging, the key biological events are validated at both protein and transcriptome levels, so that key characteristics of post-implantation embryos during gastrulation can be well reproduced. Through this model, an in vitro platform for screening drugs affecting early embryonic development can be established, providing a reference for clinical medication.

Although the present invention has been described to some extent, it is apparent that appropriate variations of all the conditions may be made without departing from the spirit and scope of the present invention. It may be understood that the present invention is not limited to the embodiments but falls within the scope of the claims, including equivalent replacements for all the elements. The specification and accompanying drawings of the present invention are illustrative and do not constitute a limitation to the claims. The protection scope of the present invention is limited by the claims and their equivalents. The specification of the present invention includes a plurality of inventive concepts, for example, “preferably”, “according to a preferred implementation”, or “optionally” indicates that the corresponding paragraph discloses a separate concept. The applicant reserves the right to file a divisional application based on each inventive concept. Herein, the feature introduced by “preferably” is only optional and should not be construed as mandatory. Therefore, the applicant reserves the right to waive or delete relevant preferred features at any time.

1. A method for constructing a gastruloid stem cell line, comprising the following steps:
(1) induction from stem cells to mesoderm:
(1-1) digesting stem cells into single cells and resuspending the cells, to obtain a cell suspension 1, wherein
the stem cells are human embryonic stem cells or human induced pluripotent stem cells, and
the human embryonic stem cells are established human embryonic stem cells derived from an embryo that is within 14 days after fertilization and that is not developed in vivo;
(1-2) centrifuging the cell suspension 1, removing a supernatant, and adding a GK15-1 medium containing a ROCK inhibitor to further resuspend cells, to obtain a cell suspension 2, wherein
in step (1-2), the cell suspension 2 and the GK15-1 medium containing the ROCK inhibitor are in the following proportion relationship: 1×10⁶ cells per 1 mL of the GK15-1 medium containing the ROCK inhibitor;
(1-3) inoculating the cell suspension 2 obtained in (1-2) into a well plate pre-coated with matrigel for culture, wherein
in step (1-3), the cell suspension 2 is inoculated in a density of 0.6-1×10⁵ cells/cm², and
in step (1-3), the culture is carried out at 37° C. under carbon dioxide in a volume concentration of 5.0%-5.2%; and
(1-4) replacing the spent medium with a GK15-1 medium on day 2 of the culture, and changing the medium daily, until nascent mesoderm-like cells are obtained;
(2) induction from the nascent mesoderm-like cells to primordial germ cell-like cells:
(2-1) digesting the nascent mesoderm-like cells obtained in (1-4) into single cells when the cells grow to 60%-90% confluency, and resuspending the cells, to obtain a cell suspension 3;
(2-2) centrifuging the cell suspension 3, removing a supernatant, and adding a GK15-2 medium containing a ROCK inhibitor to further resuspend cells, to obtain a cell suspension 4, wherein
in step (2-2), the cell suspension 3 and the GK15-2 medium containing the ROCK inhibitor are in the following proportion relationship: 1×10⁵ cells per 1 mL of the GK15-2 medium containing the ROCK inhibitor; and
(2-3) inoculating the cell suspension 4 obtained in (2-2) into a low-attachment well plate for sphere-forming culture; replacing the spent medium with a GK15-2 medium on day 2 of the culture, and changing the medium daily, until cell spheres containing primordial germ cell-like cells are obtained, wherein
in step (2-3), each well initially contains 0.5-1×10⁴ cells;
(3) cell purification:
(3-1) digesting the cell spheres obtained in (2-3) into single cells, and resuspending the cells with a GK15-2 medium, to obtain a cell suspension 5; and
(3-2) separating CD326 and CD49f double-positive cells from the cell suspension 5 obtained in (3-1), and adding a GK10 medium containing a ROCK inhibitor to resuspend the cells, to obtain a cell suspension 6, wherein
in step (3-2), 1×10⁵ cells are contained per 1 mL of the GK10 medium containing the ROCK inhibitor; and
(4) cell expansion:
inoculating the cell suspension 6 obtained in (3-2) into a well plate in which mitomycin C-treated MEFs are pre-spread as feeder cells; and replacing the medium with a fresh GK10 medium after 24 hours of culture, to obtain the gastruloid stem cell line, wherein
in step (4), the cell suspension 6 is inoculated in a density of 0.4-2×10⁴ cells/cm², and
in step (4), the culture is carried out at 37° C. under carbon dioxide in a volume concentration of 5.0%.

2. The method for constructing the gastruloid stem cell line according to claim 1, wherein
the GK15-1 medium containing the ROCK inhibitor in (1-2) comprises the following components:
80%-85% of basal medium GMEM in percentage by volume, 10%-15% of serum replacement KOSR in percentage by volume, 1% of penicillin-streptomycin double antibiotic solution in percentage by volume, 1% of 10 mM non-essential amino acids in percentage by volume, 1% of 200 mM GlutaMAX additive in percentage by volume, 1% of 100 mM sodium pyruvate additive in percentage by volume, 0.1 mM β-mercaptoethanol, 25-200 ng/mL recombinant human activin A, 1-10 μM glycogen synthase kinase-3 (GSK-3) inhibitor CHIR 99021, and 5-20 μM ROCK inhibitor;
the GK15-1 medium in (1-4) comprises the following components:
80%-85% of basal medium GMEM in percentage by volume, 10%-15% of serum replacement KOSR in percentage by volume, 1% of penicillin-streptomycin double antibiotic solution in percentage by volume, 1% of 10 mM non-essential amino acids in percentage by volume, 1% of 200 mM GlutaMAX additive in percentage by volume, 1% of 100 mM sodium pyruvate additive in percentage by volume, 0.1 mM β-mercaptoethanol, 25-200 ng/mL recombinant human activin A, and 1-10 μM glycogen synthase kinase-3 (GSK-3) inhibitor CHIR 99021;
the GK15-2 medium containing the ROCK inhibitor in (2-2) comprises the following components:
80%-85% of basal medium GMEM in percentage by volume, 10%-15% of serum replacement KOSR in percentage by volume, 1% of penicillin-streptomycin double antibiotic solution in percentage by volume, 1% of 10 mM non-essential amino acids in percentage by volume, 1% of 200 mM GlutaMAX additive in percentage by volume, 1% of 100 mM sodium pyruvate additive in percentage by volume, 0.1 mM β-mercaptoethanol, 100-500 ng/mL recombinant human bone morphogenetic protein 4, 50-200 ng/ml recombinant human stem cell factor, 1000-5000 U/mL recombinant human leukemia inhibitory factor, 50-250 ng/ml recombinant human epidermal growth factor, and 5-20 μM ROCK inhibitor;
the GK15-2 medium in (2-3) and (3-1) comprises the following components:
80%-85% of basal medium GMEM in percentage by volume, 10%-15% of serum replacement KOSR in percentage by volume, 1% of penicillin-streptomycin double antibiotic solution in percentage by volume, 1% of 10 mM non-essential amino acids in percentage by volume, 1% of 200 mM GlutaMAX additive in percentage by volume, 1% of 100 mM sodium pyruvate additive in percentage by volume, 0.1 mM β-mercaptoethanol, 100-500 ng/ml recombinant human bone morphogenetic protein 4, 50-200 ng/mL recombinant human stem cell factor, 1000-5000 U/mL recombinant human leukemia inhibitory factor, and 50-250 ng/mL recombinant human epidermal growth factor;
the GK10 medium containing the ROCK inhibitor in (3-2) comprises the following components:
80%-85% of basal medium GMEM in percentage by volume, 10% of serum replacement KOSR in percentage by volume, 2.5% of fetal bovine serum FBS in percentage by volume, 1% of penicillin-streptomycin double antibiotic solution in percentage by volume, 1% of 10 mM non-essential amino acids in percentage by volume, 1% of 200 mM GlutaMAX additive in percentage by volume, 1% of 100 mM sodium pyruvate additive in percentage by volume, 0.1 mM β-mercaptoethanol, 10 μM forskolin, 10 μM Rolipram, 50-200 ng/mL recombinant human stem cell factor, and 5-20 μM ROCK inhibitor; and
the GK10 medium in (4) comprises the following components:
80%-85% of basal medium GMEM in percentage by volume, 10% of serum replacement KOSR in percentage by volume, 2.5% of fetal bovine serum FBS in percentage by volume, 1% of penicillin-streptomycin double antibiotic solution in percentage by volume, 1% of 10 mM non-essential amino acids in percentage by volume, 1% of 200 mM GlutaMAX additive in percentage by volume, 1% of 100 mM sodium pyruvate additive in percentage by volume, 0.1 mM β-mercaptoethanol, 10 μM forskolin, 10 μM Rolipram, and 50-200 ng/mL recombinant human stem cell factor.

3. A gastruloid stem cell line, constructed by using the method according to claim 1.

4. The gastruloid stem cell line according to claim 3, wherein the gastruloid stem cell line is deposited in the China Center for Type Culture Collection, with a culture name of human gastruloid stem cell line CCRM-hGOSC-1 and an accession number of CCTCC NO. C2022114 on a deposition date of Apr. 27, 2022.

5. The gastruloid stem cell line according to claim 3, wherein the gastruloid stem cell line is deposited in the China Center for Type Culture Collection, with a culture name of human gastruloid stem cell line DYR0100-hGOSC-1 and an accession number of CCTCC NO. C2022115 on a deposition date of Apr. 27, 2022.

6. (canceled)

7. A gastruloid model, obtained through induced differentiation of the gastruloid stem cell line according to claim 3.

8. A method for constructing a gastruloid model, wherein the construction method is induced differentiation of the gastruloid stem cell line according to claim 3, and the induced differentiation is induced differentiation in vivo or induced differentiation in vitro.

9. The construction method according to claim 8, wherein the induced differentiation in vivo comprises the following steps:
resuspending the gastruloid stem cell line with a GK10 medium to obtain a cell suspension, and injecting the cell suspension into a testis of a mouse for culture for 10-20 days, to obtain the gastruloid model, wherein
the mouse is an immunodeficient mouse.

10. The construction method according to claim 9, wherein an injection amount is 2-8×10⁴ cells for one testis.

11. The construction method according to claim 8, wherein the induced differentiation in vitro comprises the following steps:
(1) digesting gastruloid stem cells into single cells when the cells grow to 60%-90% confluency, and resuspending the cells with a GK10 medium, to obtain a cell suspension 7; separating CD326 and CD49f double-positive cells from the cell suspension 7; carrying out centrifugation, resuspending the cells with an mTR medium in a concentration of 1.5-1.75×10⁴ cells/mL, and inoculating the cells into a low-attachment well plate with 6.0-7.0×10³ cells per well for culture, until the gastruloid stem cells are assembled to form a three-dimensional structure; and
(2) adding an E6BIN medium without removing the mTR medium in (1) for culture, until an amniotic cavity is formed and mesoendodermal lineage specialization is completed, to obtain the gastruloid model, wherein
in step (1) and step (2), the culture is carried out at 37° C. under carbon dioxide in a volume concentration of 5.0%.

12. The method for constructing the gastruloid model according to claim 11, wherein the mTR medium in step (1) comprises the following components: 99% of mTeSR™ 1 complete medium in percentage by volume, 1% of penicillin-streptomycin double antibiotic solution in percentage by volume, and 5-20 μM ROCK inhibitor.

13. The method for constructing the gastruloid model according to claim 11, wherein the E6BIN medium in step (2) comprises the following components: 100% of Essential 6 medium in percentage by volume, 20 ng/mL recombinant human fibroblast growth factor 2, 50 ng/mL recombinant human Noggin protein, and 5 μM IWP-2.

14. An organoid model, wherein the organoid model is obtained through culture of the gastruloid stem cell line according to claim 3 injected into an animal testis, an organoid is a tissue or organ from ectoderm, mesoderm, and endoderm, and the culture is carried out for 30-90 days.

15. A method for constructing an organoid model, comprising the following steps: resuspending the gastruloid stem cell line according to claim 3 with a GK10 medium to obtain a cell suspension, and injecting the cell suspension into a testis of a mouse for culture for 30-90 days, to obtain the organoid model, wherein
the mouse is an immunodeficient mouse.

16. The construction method according to claim 15, wherein 2-8×10⁴ cells are injected into one testis.

17. The method for constructing the organoid model according to claim 16, wherein
a neural ectoderm model, a primordial germ cell model, and/or an amniotic epithelial cell model are/is obtained on day 30 to day 40 of the culture;
a neuroepithelial cell model is obtained on day 40 to day 50 of the culture; and
an intestinal organoid model, a muscular organoid model, a cartilaginous organoid model, a neural organoid model, and/or a skin organoid model are/is obtained on day 70 to day 90 of the culture.

18. A method comprising researching mechanisms of early embryonic development of humans by using the gastruloid stem cell line according to a gastruloid model obtained through induced differentiation of the gastruloid stem cell line, an organoid model obtained through culture of the gastruloid stem cell line injected into an animal testis, an organoid is a tissue of organ from ectoderm, mesoderm, and endoderm or a tissue or organ derived from the cell line, the gastruloid model, or the organoid model, or a culture thereof.

19. A method comprising developing a diagnostic strategy and/or therapeutic strategy for a disease related to early embryonic development of humans by using the gastruloid stem cell line according to claim 3, a gastruloid model obtained through induced differentiation of the gastruloid stem cell line, an organoid model obtained through culture of the gastruloid stem cell line injected into an animal testis, an organoid is a tissue of organ from ectoderm, mesoderm, and endoderm, or a tissue or organ derived from the cell line, the gastruloid model, or the organoid model, or a culture thereof.

20. A method comprising screening, validating, evaluating, assessing, or studying a medicament in terms of efficacy for prevention and/or treatment of a disease related to early embryonic development of humans by using the gastruloid stem cell line according to a gastruloid model obtained through induced differentiation of the gastruloid stem cell line, an organoid model obtained through culture of the gastruloid stem cell line injected into an animal testis, an organoid is a tissue or organ from ectoderm, mesoderm, and endoderm, or a tissue or organ derived from the cell line, the gastruloid model, or the organoid model, or a culture thereof.
